(19) United States
(12) Patent Application Publication
(10) Pub. No.: US 2024/0366259 A1
(43) Pub. Date: Nov. 7, 2024

(54) SURGICAL DEVICES FOR SOFT TISSUE AUGMENTATION

(71) Applicant: Expand Medical Ltd., Amirim (IL)

(72) Inventors: Arie Levy, Shoam (IL); Ofek Levin, Amirim (IL)

(21) Appl. No.: 18/653,326

(22) Filed: May 2, 2024

Related U.S. Application Data
(60) Provisional application No. 63/463,786, filed on May 3, 2023.

(51) Int. Cl. A61B 17/3211; A61B 17/3209

(52) U.S. Cl. CPC A61B 17/3211; A61B 2017/32096

(57) ABSTRACT

The disclosure provides systems, methods, and devices for making attachments to soft tissue. The soft tissue attachment system uses tools that have elongated shafts useful in arthroscopic or keyhole surgeries. In the soft tissue system, a tool is taken to the site once and used to both make the hole into the tissue and simultaneously carry the anchor into the hole that is being made.

### TECHNICAL FIELD

The disclosure relates to surgical devices.

### BACKGROUND OF THE INVENTION

Injury or damage to joints in the body can be painful. Common causes of such injury include intense activity and age-related tissue deterioration. For example, ligament tears may arise from degenerative changes that occur with aging or from acute injuries during sports or other intensive activity. Treating those injuries to the musculoskeletal system is what defines the medical specialties of orthopedics and orthopedic surgery. Common orthopedic surgeries include repairs of the spine, shoulder, hand, hip, or knee, among others. For example, some people may suffer from a painful shoulder injury in the form a tear to one of a group tendons in the shoulder known as the rotator cuff. A complete tear of a rotator cuff tendon can be very painful and cause the loss of arm function. Orthopedic surgeries address those injuries. Orthopedic surgery is not without drawbacks. For example, the practical reality of surgical conditions in so-called keyhole or arthroscopic surgery means there is limited access to the surgical site.

### SUMMARY OF THE INVENTION

The disclosure provides systems, methods, and devices for making attachments to soft tissue. Devices of the disclosure are particularly useful in orthopedic surgery and particular for keyhole or arthroscopic surgeries such as for joint repair, e.g., rotator cuff repair. Using device of disclosure, a graft (or ligament, suture, tendon, etc.) may be attached to soft tissue. The soft tissue attachment system includes a delivery tool with a blade extending therefrom and an implantable soft tissue anchor carried on the blade. The anchor preferably has a leg extending forward from a base. The leg of anchor is carried against the blade of the delivery tool, such that the tool blade plus the leg of the anchor cooperate to the form one tissue-piercing element. The blade of the tool extends further forward than the leg of the anchor and the leading point of the blade is very thin and sharp, e.g., sharpened metal such as a high grade stainless steel. The leg of the anchor is also sloped or pointed, and preferably has one or more barbs sticking out. By pushing the system into soft tissue, the blade slices open the tissue and carries the leg of the anchor into newly-formed hole. Preferred embodiments have tack anchors, with a single leg that extend forward to be pushed into tissue. The leg have barbs that extend outwards from the anchor to grip into the tissue. A base extends laterally from a proximal end of the leg and operates to strap a graft (or ligament) down to the tissue and hold it in place. The blade(s) of the delivery tool can be retracted, leaving the anchor embedded in, and securely attached to, the soft tissue.

Preferred embodiments of the delivery tool have an extended sleeve slidably disposed over the elongated shaft. The sleeve can be translated (relative to the handle, shaft, and delivery blade with its sharp tip) between a proximal and distal position. The sleeve has a tapered or conical distal tip with a slot cut in from the distal end, dividing the sleeve into two semi-conical or partially spherical counter parts or two sides of a cover that can bend apart from each other (elastically) to allow the attachment device to push distally out of the sleeve when the sleeve is slid back, proximally, along the delivery tool.

Most preferably, the blade, shaft, and handle are attached together into a single subassembly with no moving parts. A sleeve is slidably disposed over the shaft. The sleeve has a split conical tip present as two half covers that can bend away from each other. The surgeon can pull a switch on the handle to slide the sleeve back, causing the conical tip to open it passes over the base of the implantable attachment device and then close behind the base. The surgeon pushes the implantable attachment device into tissue specifically by pushing forward by the handle. The shaft, where it transitions to the sharp blade, presents one or more steps (e.g., metal shoulders of the shaft) that push the implantable attachment device in the distal direction, through the graft and into tissue. After the shaft has been used to drive the attachment device through a graft and into soft tissue, the closed conical tip sitting behind the base can be used to apply counterpressure to the base while the blade is withdrawn from the from the tissue. The counterpressure keeps the implanted attachment device from wiggling or moving in a manner that would compromise the attachment strength.

The attachment is also very strong due to the nature and configuration of the barbs of the implantable attachment device. The barbs extend in opposed lateral directions away from the leg of the attachment device and away from each other. While multi-leg implantable attachment devices are included in the scope of this disclosure, the ability to have multiple barbs point away from each other and away from the legs of the device mean that the disclosed implantable attachment devices form strong enough attachments in soft tissue, even with only one leg. Thus, preferably, the attachment device includes only one leg and two laterally opposed barbs pointing outward from the leg. Notably, no part of the delivery tool surrounds the outside of the attachment device leg. I.e., the delivery tool has a flat blade that lies only along one side of the leg.

The surgeon can navigate a delivery tip of the delivery tool through an incision. The attachment device is carried on the blade within, and covered by, the split tip of the conical or tapered sleeve. When the delivery tip is positioned over a target attachment site on soft tissue (e.g., where a graft, tendon, or ligament is to be attached to soft tissue underneath), the surgeon uses a thumb to pull back on a trigger on the delivery tool, drawing the sleeve backwards, exposing the attachment device therein, effectively sliding the attachment device out of the distal tip of the sleeve.

In fact, when the base of the attachment device slides beyond a distal tip of the sleeve, the two sides of the cover (i.e., the split conical or tapered end of the sleeve) elastically return to the closed position behind the back of the base of the attachment device. In that disposition, the delivery tool can be used to push the attachment device into soft tissue. By pushing forward with the handle, the closed sleeve end pushes the attachment device forwards, while the sharpened tip of the sharp blade (preferably stainless steel) slices into the tissue and initiates entry into the tissue, carrying the attachment device through any graft or ligament and into the tissue.

With the attachment device properly attached, the surgeon can place a finger on a hook or other ridge or grip of the handle of the delivery tool and use a thumb on the trigger to simultaneously apply forward pressure with the sleeve while withdrawing the delivery tool from the site. The distal tip of the sleeve provide counterpressure on the attachment device while the delivery tool is withdrawn from the site. The counterpressure negates any tendency for the sharp blade to try to withdraw the attachment device by friction forces. Instead, the counter pressure holds the attachment device stably in the target while the device is removed.

Also, pushing the sleeve forward while gently pulling the handle back (e.g., using a thumb on the trigger and a finger on the hook) pulls the sharp blade back inside of the sleeve so that when the delivery tool is carried away from the site, removed through the incision, and potentially handled by the surgeon and attending staff, the sharp blade is fully covered by the sleeve.

The soft tissue attachment system uses tools that have elongated shafts extending from handles. Those shafts are designed for ease of insertion through a minimally invasive surgical incision or trocar, such as those made and used in arthroscopic or keyhole surgeries. Thus these anchoring system are useful during joint repair, to bring anchors into the surgical site and both anchor a graft to bone (e.g., using the bone anchor system) and then also attach the graft to soft tissue in the area. In each case, the tool is taken to the site once and used to both make the hole into the bone or tissue and simultaneously carry the anchor into the hole that is being made. Using systems of the invention, the surgeon does not need to make pilot holes or cuts first, and then return with a separate tool to place the anchor or attachment device. This avoids problems with misalignment and risky do-overs. Also, the attachment devices of the disclosure provide secure, durable attachment.

Aspects provide a system for fixing soft tissue. The system include a delivery tool with an elongated shaft extending from a handle, at least a first sharp blade extending distally from a distal end of the elongated shaft, and an attachment device positioned on the first sharp blade. The attachment device includes a leg extending from a proximal portion to a distal tip that is shaped with a taper and a base connected to the proximal portion of the leg. The base extends substantially perpendicular to the leg and has an open portion adjacent the proximal portion of the leg that is configured to allow a sharp blade to extend through the base and along a side of the leg. In the system, the first sharp blade extends through the open portion of the base.

The first sharp blade may be a substantially flat piece (e.g., of sharpened stainless steel) with opposed first and second surfaces extending distally to a sharpened piercing tip. Preferably, the sharpened piercing tip of the first sharp blade extends distally beyond the distal tip of first leg of the attachment device. The leg of the attachment device may lay flat against a surface along a side of the first sharp blade. When the first sharp blade is pushed distally into soft tissue, a tip of the first sharp blade pierces a hole into the tissue and the distal tip of the leg is pushed into the hole.

In preferred embodiments, the delivery tool comprises a sleeve disposed over the shaft. The sleeve is slidable between a proximal and a distal position. When the sleeve is in the distal position, the sleeve covers the first sharp blade. The sleeve may have a tapered or conical tip, with a slit into the tapered conical tip that divides the tapered conical tip into two half covers that can bend away from each other.

In use, when the attachment device is positioned on the blade, the sleeve can be retracted, by a trigger on the handle, to allow the attachment device and blade to extend beyond the conical tip, wherein the conical tip closes behind a base of the attachment device and may be used to push the attachment device and blade into tissue. The sleeve protects tissue and any cannula from the sharp blade during insertion of the delivery tool into a surgical site; the conical tip facilitates smooth and easy insertion through the incision; after the attachment device has been inserted into tissue, the conical tip applies counterpressure to a base of the attachment device to hold the attachment device still in tissue while the sharp blade is retracted; and/or the conical tip covers the sharp blade during removal of the delivery tool from the site to protect surrounding tissue and personnel.

Preferably, the first sharp blade, the shaft, and the handle are fixedly attached to each other to form one sub-device with no moving parts. The sleeve can translate over the shaft of the sub-device. The sleeve may present a trigger extending from and operable from the handle to slide the sleeve distally and proximally. In some embodiments, the trigger naturally rests in distal and proximal cutouts along a slot in the handle, and a surgeon can elastically deform the trigger out of the cutouts to slide the sleeve.

In certain embodiments, the first sharp blade and the shaft are stainless steel, while the sleeve and the trigger are an elastic or plastic material. The (implantable) attachment device may be made of an absorbable material, a bioabsorbable material, or a biological material.

In certain aspects, the disclosure provides a surgical attachment device that includes at least a first leg extending from a proximal portion to a distal tip that is shaped with a taper and a base connected to the proximal portion of the first leg. The base extends substantially perpendicular to the first leg. The base comprises an open portion adjacent the proximal portion of the leg, and the open portion is configured to allow a piercing tool to extend through the base and along a side of the leg. The first leg may include a barb protruding from the first leg, e.g., in a direction that does not overhang the open portion of the base. The taper of the distal tip may extend smoothly over the barb. Preferably, the first leg has at least one substantially planar surface along a length of the first leg, and the open portion has an inward facing flat portion, such that the substantially planar surface and the flat portion define a continuous flat surface adjacent an open space that allows a flat blade to slide proximally and distally against the continuous flat surface. The device may include one or more barbs configured to allow the first leg to be pushed in a distal direction into soft tissue and to impede the first leg from being withdrawn in a proximal direction out of the soft tissue. Preferably, the one or more barbs do not extend into the open space through which the flat blade may slide proximally and distally against the continuous flat surface.

In some embodiments, the device includes a second leg extending from the base to a second distal tip that is shaped with a second taper, the second leg being substantially parallel to the first leg. The base may include a second open portion adjacent a proximal portion of the second leg. The distal tip may terminate at a sharp point with the taper extending smoothly from the sharp point over a first barb protruding from the first leg in a direction that does not overhang the open portion of the base, and the second distal tip may terminate at a second sharp point with the second taper extending smoothly from the second sharp point over a second barb protruding from the second leg in a direction that does not overhang the second open portion of the base. Preferably, the first barb and the second barb do not extend medially between the first leg and the second leg (i.e., the barbs point outwards to afford greater purchase within soft tissue).

In certain embodiments, the first leg and the first open portion define a flat surface along which a delivery blade may slide through the base and the second leg and the second open portion may define a second flat surface along which a second delivery may slide through the base. Preferably a proximal side of the base comprises a pushable surface by which the first leg and the second leg may be pushed into soft tissue, wherein the first distal tip and the second distal tip are sharpened to pierce the soft tissue. A distal side of the base, between the first leg and the second leg, presents a substantially flat surface operable to hold a graft or ligament against soft tissue when the first leg and the second leg are pushed into the soft tissue. The device may be made of a polymer material that exhibits deformability. The base functions as a cap that may be broader than the first leg and operable to hold a graft against tissue and wherein the open portion is an aperture through the cap.

In related aspects, the invention provides a system for fixing soft tissue. The system includes the attachment device described above along with a delivery tool with an elongated shaft extending from a handle and at least a first sharp blade extending distally from a distal end of the elongated shaft. The attachment device may be positioned on the first sharp blade, with first sharp blade extending through the open portion of the base.

In some embodiments, the first sharp blade comprises a substantially flat piece with opposed first and second surfaces extending distally to a sharpened piercing tip. The sharpened piercing tip of the first sharp blade preferably extends distally beyond the distal tip of first leg of the attachment device. The first leg of the attachment device may lie flat against a surface along a side of the first sharp blade. When the first sharp blade is pushed distally into soft tissue, a tip of the first sharp blade pierces a hole into the tissue and the distal tip of the first leg is pushed into the hole. Preferably, after the first sharp blade is pushed into tissue to carry the attachment device into the tissue, the first sharp blade can be retracted into the shaft, withdrawing the first sharp blade proximally through the open portion of the base of the attachment device. When the blade is withdrawn proximally, one or more barbs on the attachment device may hold the attachment device in the tissue.

In some embodiments, the attachment device comprises a second leg extending from the base to a second distal tip that is shaped with a second taper, the second leg being substantially parallel to the first leg, and the delivery tool has a second blade extending through a second open portion of the attachment device and lying against the second leg.

When the attachment device is mounted on the delivery tool, the first blade may present a first sharpened point extending distally from the distal tip of the first leg while the second blade presents a second sharpened point extending distally from a second distal tip of the second leg. Preferably barbs extend outward from the first leg and the second leg to grip soft tissue at locations that are not medial between the first and second leg. The legs and respective open portions of the base may define flat surface along which the blades lie. Preferably the first leg and first sharp blade cooperate to define a tissue-piercing element terminating at a sharpened tip of the first sharp blade, and the second leg and the second blade cooperate to a define a second tissue-piercing element terminating a second sharpened tip of the second blade.

In some embodiments, the handle comprises a spring biasing the first sharp blade in a distal position and a switch operable to restrain the first sharp blade in a proximal position. When the first sharp blade is in the proximal positioned, the first sharp blade may be contained within and covered by a distal portion of the shaft.

Aspects of the disclosure provide methods for attaching to soft tissue. The methods involve using the system or the attachment devices described above. A surgeon holds the delivery tool by the handle, with the soft tissue attachment device mounted on the blade. The surgeon places the elongated shaft through a surgical incision or cannula, e.g., in arthroscopic or keyhole surgery such as for joint repair, including for example, rotator cuff repair. The sharp blade extends distally from a distal end of the elongated shaft, through

the open portion on the base of the attachment device and extending along the leg of the attachment device.

The surgeon navigates a delivery tip of the delivery tool through the incision. The attachment device may be carried on the blade within, and covered by, a split tip of the conical or tapered sleeve. In certain embodiments, when the delivery tip is positioned over a target attachment site on soft tissue (e.g., where a graft, tendon, or ligament is to be attached to soft tissue underneath), the surgeon uses a thumb to pull back on a trigger on the delivery tool, drawing the sleeve backwards, exposing the attachment device therein, effectively sliding the attachment device out of the distal tip of the sleeve. In those embodiments, when the base of the attachment device slides beyond a distal tip of the sleeve, the two sides of the cover (i.e., the split conical or tapered end of the sleeve) elastically return to the closed position behind the back of the base of the attachment device. In that disposition, the delivery tool can be used to push the attachment device into soft tissue. By pushing forward with the handle, the closed sleeve end pushes the attachment device forwards, while the sharpened tip of the sharp blade (preferably stainless steel) slices into the tissue and initiates entry into the tissue, carrying the attachment device through any graft or ligament and into the tissue.

With the attachment device properly attached, the surgeon can place a finger on a hook or other ridge or grip of the handle of the delivery tool and use a thumb on the trigger to simultaneously apply forward pressure with the sleeve while withdrawing the delivery tool from the site. The distal tip of the sleeve provide counterpressure on the attachment device while the delivery tool is withdrawn from the site. The counterpressure negates any tendency for the sharp blade to try to withdraw the attachment device by friction forces. Instead, the counter pressure holds the attachment device stably in the target while the device is removed.

In methods of the invention, pushing the sleeve forward while gently pulling the handle back (e.g., using a thumb on the trigger and a finger on the hook) pulls the sharp blade back inside of the sleeve so that when the delivery tool is carried away from the site, removed through the incision, and potentially handled by the surgeon and attending staff, the sharp blade is fully covered by the sleeve.

The surgeon uses the sharp blade, and then the pointed tip of the attachment device, to pierce through any graft or ligament that is to be attached to soft tissue and also into the soft tissue. The distal tip (of the leg of the implantable soft tissue attachment device) terminates at a sharp point. The blade(s) of the delivery tool pierce any graft and the soft tissue. The base of the implantable soft tissue attachment device is broader than the first leg and holds a graft or ligament against tissue. Once the attachment device is in tissue, the sharp blade is retracted, and the delivery tool is removed from the surgical site. In some embodiments, the open portion is an aperture through the cap and has benefits both during delivery (the blade extends through the aperture) and after surgery, where tissue may grow into the opening increasing fixation.

In some embodiments, which may optionally include both first and second sharp blades (e.g., for attachment devices with respective first and second legs), when the first sharp blade is pushed distally into soft tissue, a tip of the first sharp blade pierces a hole into the tissue and the distal tip of the first leg is pushed into the hole (the same steps may be happening with a second blade and second leg). After the first sharp blade is pushed into tissue to carry the attachment device into the tissue, the first sharp blade can be retracted into the shaft, withdrawing the first sharp blade proximally through the open portion of the base of the attachment device. When the blade is withdrawn proximally, one or more barbs on the attachment device hold the attachment device in the tissue. When the attachment device is mounted on the delivery tool, the first blade presents a first sharpened point extending distally from the distal tip of the first leg, and the second blade presents a second sharpened point extending distally from a second distal tip of the second leg.

Aspects of the invention provide a system for fixing soft tissue. The system includes a delivery tool with an elongated shaft extending from a body; a handle and a trigger extending from the body; a sharp blade extending distally from a distal end of the elongated shaft; an attachment device disposed on the blade; and a sleeve extending from the body and covering the sharp blade and the attachment device. The attachment device has a tapered leg extending from a proximal portion to a distal tip, and a base connected to the proximal portion of the leg, the base extending substantially perpendicular to the tapered leg. The base comprises an open portion adjacent the proximal portion of the leg, and the sharp blade extends through the base and along a side of the tapered leg when the attachment device is positioned on the sharp blade. Squeezing the trigger towards the handle pulls the sleeve in a proximal direction towards the handle, exposing the sharp blade and the attachment device. In some embodiments, the trigger comprises gears engaged with a crank shaft hub that is connected to the sleeve via a connecting rod, whereby squeezing the trigger rotates the crank shaft hub, pulling the sleeve via the connecting rod to translate over, and uncover, the sharp blade. The crank shaft hub may be engaged with a ratchet mechanism, such that squeezing the trigger a second time pushes the sleeve via the connecting rod to cover the sharp blade. In some embodiments, the attachment device comprises a sloped face that minimizes for required for the attachment device to penetrate soft tissue. Preferably the tapered leg is wider towards the base of the attachment device than near the tip.

In certain embodiments, the sleeve comprises a conical tip and a slit into the conical tip, dividing the conical tip into two half covers that can bend away from each other, wherein translating the sleeve to a proximal position on the delivery tool causes the conical tip to open as the conical tip passes over the base of the attachment device and to close behind the base, wherein after the shaft has been used to drive the attachment device through a graft and into soft tissue, the closed conical tip sitting behind the base can be used to apply counterpressure to the base while the blade withdrawn from the attachment device.

The attachment device may include at least two laterally opposed barbs pointing outward. In preferred embodiments, the attachment device has only the one leg.

The sharp blade may have a substantially flat piece with opposed first and second surfaces extending distally to a sharpened piercing tip, wherein the sharpened piercing tip of the sharp blade extends distally beyond the distal tip of first leg of the attachment device. In certain embodiments, the sleeve includes a static comb with one or more steps that can push the attachment device in distal direction, wherein the static comb pushes the attachment device into tissue during surgery. A distal portion of the static comb may have a notch that engages with the base of the attachment device when the attachment device is positioned at a distal end of the sharp blade. Preferably, the static comb is in a fixed position with respect to the elongated shaft, such that the sleeve translates with respect to the static comb. The delivery tool may further include a dynamic comb fixed to the sleeve, in which the dynamic comb pushes pre-loaded attachment devices in a distal direction down the sharp blade to position a subsequent device in position for delivery.

In operation, when the sharp blade is pushed distally into soft tissue, a tip of the sharp blade pierces a hole into the tissue and the distal tip of the leg is pushed into the hole. The sharp blade and the elongated shaft may be stainless steel. The sleeve and the trigger may be an elastic or plastic material. The attachment device may be an absorbable material, a bioabsorbable material, or a biological material.

### BRIEF DESCRIPTION OF DRAWINGS

FIG. 1 diagrams steps of a method 101 for attaching to soft tissue.

FIG. 2 shows an attachment device 1201.

FIG. 3 shows a back view of the device 1201.

FIG. 4 shows the device 1201 sitting on a sharp blade 1301.

FIG. 5 shows a back, blade side of the delivery tip of a delivery tool.

FIG. 6 shows a delivery tool 601 for delivering a soft-tissue fastener.

FIG. 7 is a detail view of the delivery tip.

FIG. 8 shows the delivery tool 601 with the surgical attachment device.

FIG. 9 shows the attachment device 1201, having been extended from the delivery tip.

FIG. 10 is a cutaway view of the handle.

FIG. 11 is a detail view of the switch.

FIG. 12 begins a series and shows a method of attaching a graft 701 to soft tissue.

FIG. 13 shows that the delivery tip 613 is near the graft 701.

FIG. 14 shows the attachment device 1201 being used to pierce or spear the graft.

FIG. 15 shows the base 1205 of the attachment device.

FIG. 16 illustrates the delivery tip 613 being withdrawn from the graft 701.

FIG. 17 is a detail view of the delivery tip 613 being withdrawn from the graft 701.

FIG. 18 shows an implantable soft-tissue attachment device 201.

FIG. 19 shows the attachment device 201 mounted on a sharp blade 307.

FIG. 20 shows a delivery tool 301 for the soft tissue attachment device 201.

FIG. 21 is a detail view of the delivery tip 501.

FIG. 22 shows mechanisms of the delivery tool 301.

FIG. 23 is a detail view of the switch 335.

FIG. 24 shows the mechanism in the handle.

FIG. 25 is a detail view of the blades emerging from the shaft.

FIG. 26 shows the switch operating to release the blades.

FIG. 27 shows the device 301 with the blades fully extended.

FIG. 28 is a detail view of the delivery tip 501.

FIG. 29 shows another single leg embodiment of a soft tissue attachment device 1411.

FIG. 30 shows a piercing tool 1401 extended through the base of an attachment device.

FIG. 31 shows the soft tissue attachment device 1411 on the piercing tool 1401.

FIG. 32 shows a distal tip of a multi-anchor delivery tool 3200.

FIG. 33 shows a shaft 3203 for the delivery tool 3200.

FIG. 34 shows the multi-anchor delivery tool 3200 in use.

FIG. 35 shows a tapered and shaped version of an attachment device.

FIG. 36 shows a “pistol grip” version of a delivery tool.

FIG. 37 illustrates a first step in operation of the pistol-grip delivery tool.

FIG. 38 is a close-up of the delivery tip of the delivery tool.

FIG. 39 shows trigger action.

FIG. 40 illustrates a second pull (compression) of the trigger.

FIG. 41 gives a detail view of the delivery tip.

FIG. 42 is a cutaway view from a first side of the delivery tool.

FIG. 43 shows the displacement of shaft when the trigger is squeezed.

FIG. 44 is a cutaway view from a second side of the delivery tool.

FIG. 45 is a detail view showing the trigger gear engaged with the ratchet gear.

FIG. 46 is a perspective cutaway view of a delivery tip of a delivery tool.

FIG. 47 is a cutaway side view through the delivery tip.

FIG. 48 shows the blade and the first attachment device being uncovered.

FIG. 49 shows a one-at-a-time delivery mechanism for a delivery tool of the disclosure.

FIG. 50 is a cutaway side view through the one-at-a-time delivery mechanism.

FIG. 51 shows delivery via the one-at-a-time delivery mechanism.

FIG. 52 shows a tapered insertion sleeve for a delivery tool of the disclosure.

### DETAILED DESCRIPTION OF THE INVENTION

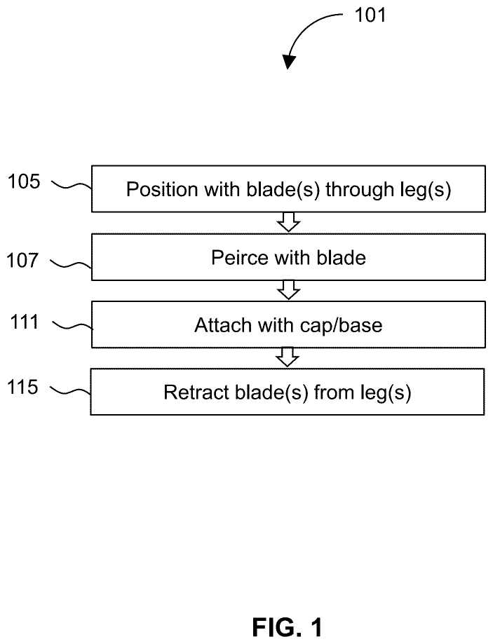

FIG. 1 diagrams steps of a method 101 for attaching a graft, ligament, tendon, or other material to soft tissue. The method includes using a delivery tool with an elongated shaft extending from a handle and at least one sharp blade extending distally from a distal end of the elongated shaft with a tissue attachment device disposed on the sharp blade. The tissue attachment device has at least a first leg extending from a proximal portion to a distal tip that is shaped with a taper and a base connected to the proximal portion of the first leg. The base extends like a cap away from the leg and will hold the graft or ligament to the soft tissue. The attachment device disposed on the blade is positioned 105 against the soft tissue. A pointed tip of blade extends beyond the distal end of the leg of the attachment device. The pointed tip of the blade pierces 107 the tissue (and the graft or ligament to be attached to the tissue). The attachment device is pushed distally into the tissue until it bottoms out with the base or cap attaching 111 the graft or ligament to the tissue. The, the blade can be retracted 115 from the leg of the attachment device.

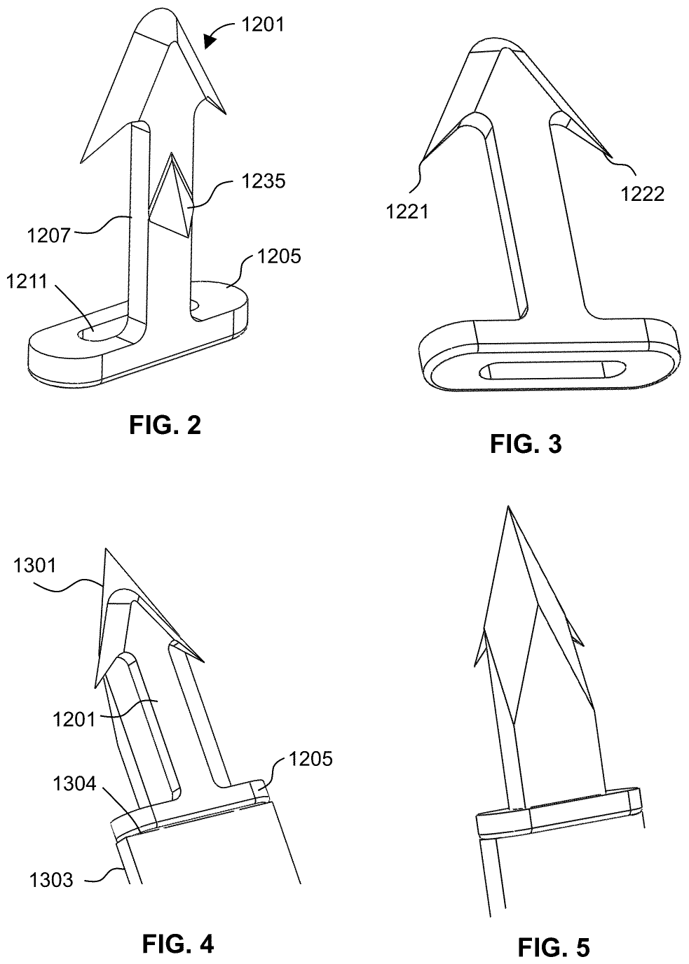

FIG. 2 shows a front view of a single arm embodiment of a surgical attachment device 1201 (aka tack, anchor, or fastener). The attachment device 1201 a leg extending 1207 from a proximal portion to a distal tip that is shaped with a taper. A base 1205 is connected to the proximal portion of the first leg 1207. The base 1204 extends substantially perpendicular to the first leg 1207. The base 1205 comprises an open portion 1211 adjacent the proximal portion of the leg 1207. The open portion 1211 allows a piercing tool with a blade 1301 to extend through the base and along a side of the leg. the attachment device comprises an absorbable material, a bioabsorbable material, or a biological material. The attachment device 1201 may be made of an absorbable polymer (e.g. PEEK, POM, Nylon, HDPE), bioabsorbable polymer (e.g. PLA, PGA, PDS, PLGA), or a biological metrical such as collagen or bone.

FIG. 3 shows a back view of the device 1201. The device 1201 has first and second opposite lateral hooks 1221, 1222 emerging at the end of the leg 1207. This design can result in better attachment strength and stability. In addition, since the arm is essentially flat along its entire length, the penetration profile is also better.

FIG. 4 shows the device 1201 sitting on a blade 1301 extending distally from a distal end of an elongated shaft 1303 of a delivery tool 1300 (which preferably has a single blade 1301 but otherwise has all elements in common with delivery tool 301 in FIG. 11A). The device 1201 is a preferred embodiment for attaching tendon to soft tissue. In use, the attachment device 1201 is positioned on the blade 1301, with blade 1301 extending through the open portion 1211 of the base 1205. The blade 1301 extends from the shaft 1303. Where the shaft 1303 transitions to the blade 1301, the shaft has one or more steps 1304 that can push against the base 1205 of the implantable attachment device 1201.

The device 1201 (e.g., tack, anchor, or fastener) may include one more additional perpendicular hook 1235, which provides additional attachment strength. In preferred embodiments, the perpendicular hook 1235 is located below (i.e., “offset” from) the lateral hooks 1221, 1222. The offset of the perpendicular hook is advantageous because penetration of the device into soft tissue is easier (than if the perpendicular hook 1235 were adjacent the lateral hooks 1221, 1222) since the first two hooks 1221, 1222 penetrate first and then the perpendicular hook 1235 enters the soft tissue. The offset configuration also creates additional attachment to multiple layers of tissue, therefore prevent movement. The offset configuration is beneficial because the connective tissue to which attachment is preferred may be present at different depths (e.g., tougher or more sinewy parts may lie within the soft tissue at various deeper and shallower depths), so the offset configuration increase a chance of one or more hooks connected into stronger connective tissue.

Similarly, the two lateral hooks 1221, 1222 are preferably slightly offset in order to achieve a better penetration profile and more distributed holding at the tissue.

Thus it can be seen that the device 1201 may have multiple hooks 1221, 1222, 1235 that engage with tissue at multiple locations, depths, and directions, providing for very strong and reliable anchoring to the soft tissue.

FIG. 5 shows a back, blade side of the delivery tip of the tool with attachment device 1201 positioned thereon.

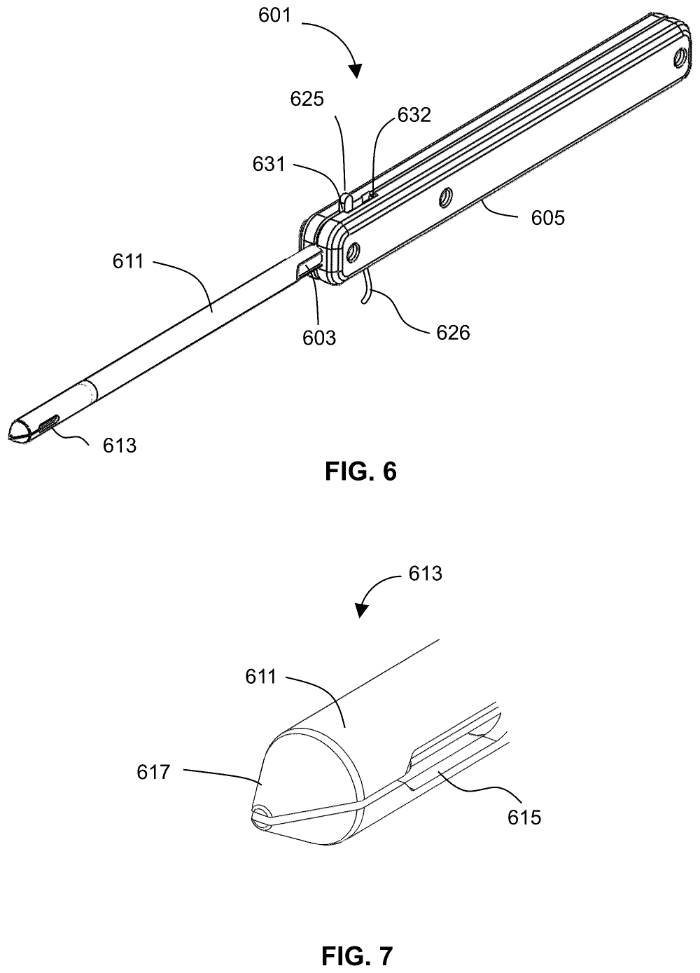

FIG. 6 shows a delivery tool 601 for delivering a soft-tissue fastener such as the surgical attachment device 1201. The delivery device 601 includes a handle 605 with a shaft 603 extending therefrom. The shaft is covered by a sleeve 611. The sleeve 611 extends distally to a sloped delivery tip 613. The sleeve 611 is preferably slidably disposed over the elongated shaft 603. The sleeve 611 can be translated (relative to the handle 605, shaft, and delivery tip) between a proximal and distal position. The delivery tool 601 includes a switch 625 extending from a portion of the sleeve 611 and optionally a hook 626 or other grip mechanism extending from the handle 605. A surgeon can translate the sleeve between proximal and distal dispositions by operating the switch 625 between a first, distal cutout 631 and a second, proximal cutout 632.

FIG. 11 is a detail view of the switch 625 resting in the second, proximal cutout 632

FIG. 7 is a detail view of the delivery tip 613 of the sleeve 611. The distal end of the delivery tip is split with a slot that separates two semi conical covers 617. Proximal from the semi conical convers 617 are openings 615. The openings allow the two opposed covers 617 to bend away from one other. The covers are elastically biased into the depicted configuration but can be bent away from one other. The sleeve 611 can thus be described as having a tapered or conical distal tip with a slot cut in from the distal end, dividing the sleeve into two semi-conical or partially spherical counter parts or two sides of a cover 617 that can bend apart from each other (elastically) to allow the attachment device to push distally out of the sleeve when the sleeve is slid back, proximally, along the delivery tool.

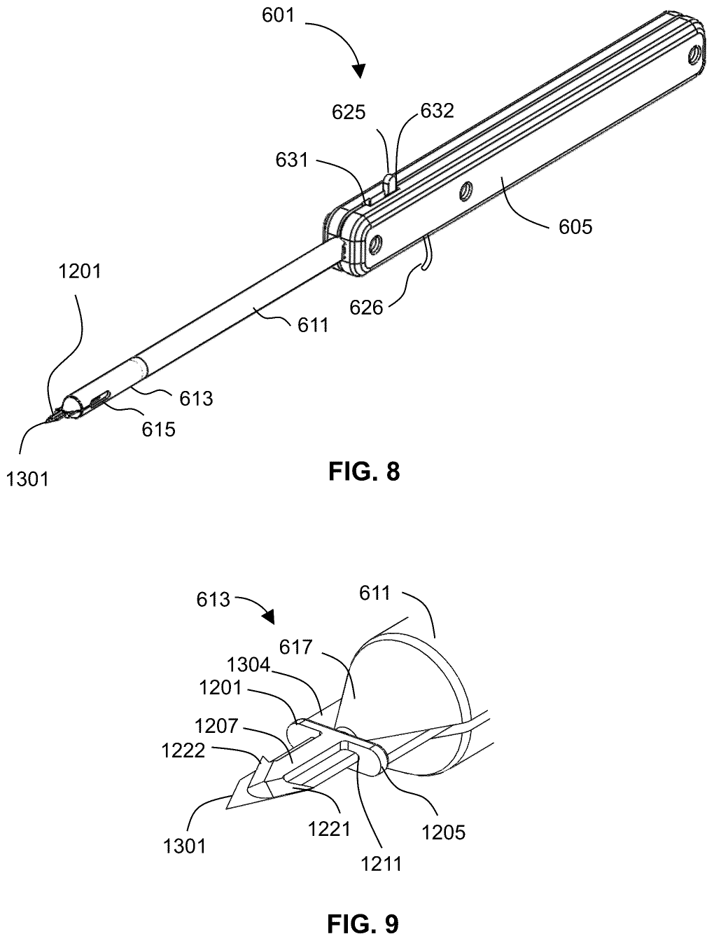

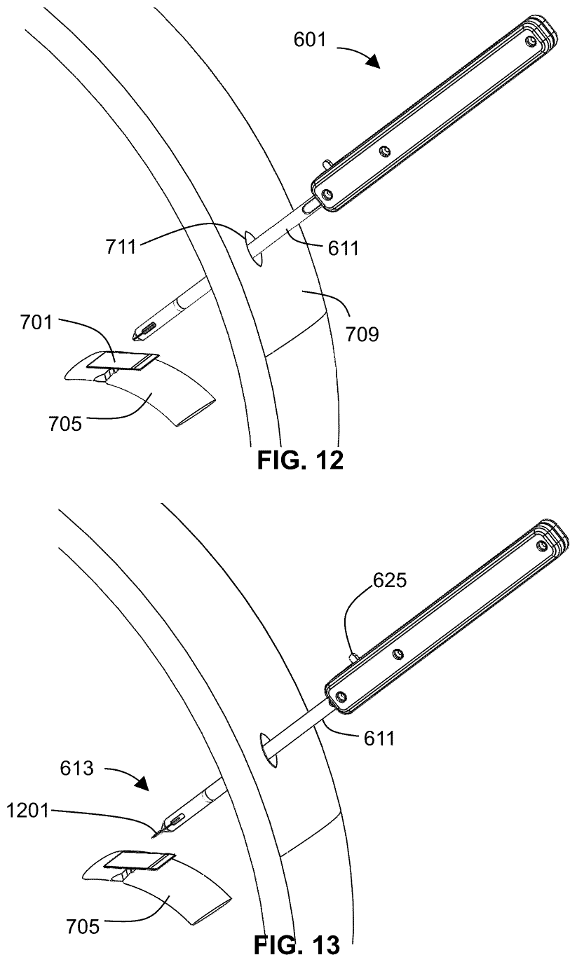

FIG. 8, FIG. 9, FIG. 12, and FIG. 15 when viewed and considered together illustrate important features of the delivery tool 601.

FIG. 8 shows the delivery tool 601 with the surgical attachment device 1201 extended out from the delivery tip 613 of the sleeve 611. Together, the delivery tool 601 and the surgical attachment device 1201 provide a system for fixing soft tissue. The distal-most point of the delivery tool 601 is a sharp blade 1301 that bases through the base 1205 of, and extends beyond a tip of, the attachment device 1201.

The switch 625 is preferably formed as a monolithic portion of the sleeve 611. The switch 625 can be deformed around an axis of the shaft 603 by a few millimeters to pull the switch 625 out of, or return the switch 625, back into the first, distal cutout 631 and the second, proximal cutout 632. The sleeve 611 and the switch 625 are made of an elastic material that biased into the cutouts. To slide the sleeve 611 forward or backward, the surgeon temporarily deforms the switch out of the cutouts and slides.

FIG. 9 shows the attachment device 1201 (e.g., tack, anchor, or fastener), having been extended from the delivery tip. Notably, the attachment device 1201 is carried within the delivery tip 613 prior to delivery. When the attachment device 1201 is inside of the delivery tip 613, the first and second lateral hooks 1221, 1222 and the base 1205 may be accommodated by the openings 615. When the sleeve 611 is retracted to the proximal position, the covers 617 spread apart to allow the base 1205 of the attachment device 1201 to pass out of delivery tip 613, while still sitting over a blade 1301 extending from the shaft 603. As shown in the image, after the attachment device 1201 is extended past a distal terminus of the sleeve 611, the covers 617 return to their resting position behind the extended base 1205. Where the shaft 1303 transitions to the blade 1301, the shaft has one or more steps 1304 that can push against the base 1205 of the implantable attachment device 1201. Due to the way that the steps 1304 push against the base 1205 of the implantable attachment device 1201, pushing the delivery tool 601 in a distal (forward) direction will push the attachment device 1201 forward.

As can be seen in the depiction, the base 1205 comprises an open portion 1211 adjacent the proximal portion of the leg 1207. The open portion 1211 allows a piercing tool with a blade 1301 to extend through the base and along a side of the leg.

In fact, when the base 1205 of the attachment device slides beyond a distal tip of the sleeve, the two sides of the cover 617 (i.e., the split conical or tapered end of the sleeve) elastically return to the closed position behind the back of the base 1205 of the attachment device. FIG. 9 clearly shows sharp blade 1301 extending past a distal tip of the attachment device 1201.

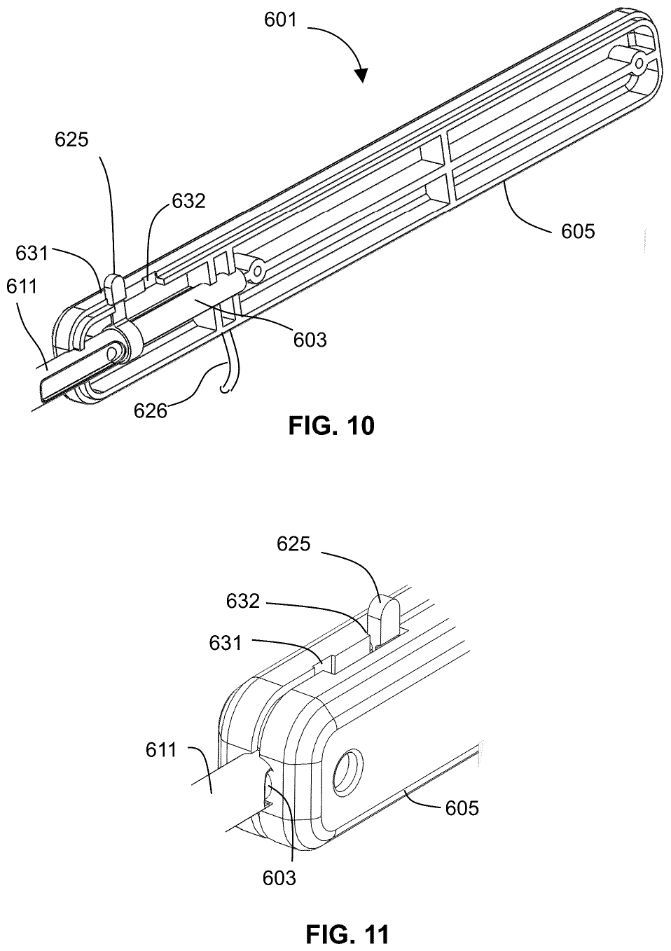

FIG. 10 is a cutaway view of the handle showing the shaft 603 mounted therein. The sleeve 611 has a switch 625 that can be used to slide the sleeve between two positions, distal and proximal, relative to the handle 605. The switch 625 can rest in a first, distal cutout 631 or, when the sleeve 611 is slid to the proximal position, the switch can rest in a second, proximal cutout 632. A preferred embodiment of the device 601 is illustrated here. The blade 1301 (shown in FIG. 9) and the shaft 603 are preferably made of stainless steel and welded together. The shaft 603 is attached to the handle 605, which has the hook 626 or other grip mechanism. The blade 1301, the shaft 603, the handle 605, and the hook 626 are preferably one device with no moving parts within that combination of elements. Only the sleeve 611 and the trigger 625 move relative to that combination.

FIG. 11 is a detail view of the switch 625 resting in the second, proximal cutout 632 in the handle 605 of the device 101, holding the sleeve 611 in the proximal position, revealing on a very minor portion of the shaft 603. The switch 625 and the sleeve 611 can move relative to the handle, which is fixed in attachment to the shaft 603 and blade 1301.

FIG. 12 begins a series and shows a method of attaching a graft 701 to soft tissue 705. A method begins by passing the sleeve 611 through an incision 711 in the tissue 705. The surgeon can navigate a delivery tip of the delivery tool through an incision. The attachment device is carried on the blade within, and covered by, the split tip of the conical or tapered sleeve. The sleeve is held in its distal position by virtue of the switch 625 setting in the first, distal cutaway 631. While not visible, the attachment device 1201 (e.g., tack, anchor, or fastener) is being carried within the delivery tip 613 of the device 301.

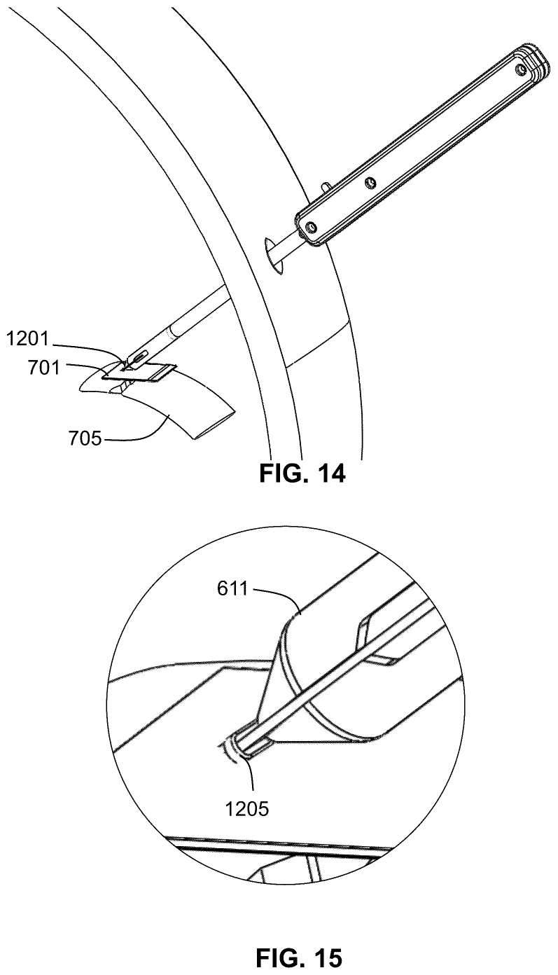

Notably, during the delivery, the blade 1301 and the attachment device 1201 are held within and covered by the sleeve 611. This holding and covering provides at least two benefits. First, because the attachment device 1201 is sharp and the blade 1301 is preferably very sharp stainless steel, the covering protects tissue surrounding the surgical site and the incision and also protects the surgeon and attending staff. Second, the covering holds the attachment device 1201 in position in the delivery tip 613 until the blade 1301 is speared through the graft 701 and into the tissue 705. Until the spearing action (shown in FIG. 14) the attachment device 1201 is covered and protected from getting dislodged during handling and setup for a surgical procedure.

FIG. 13 shows that when the delivery tip 613 is near the graft 701, the surgeon can retract the sleeve 611, but pulling the switch back to the second, proximal cutaway 632. When the delivery tip is positioned over a target attachment site on soft tissue (e.g., where a graft, tendon, or ligament is to be attached to soft tissue underneath), the surgeon uses a thumb to pull back on a trigger on the delivery tool, drawing the sleeve backwards, exposing the attachment device therein, effectively sliding the attachment device out of the distal tip of the sleeve. When the sleeve 611 is retracted to the proximal position, the delivery tip 613 opens to allow the attachment device 1201 to pass out of delivery tip 613, while still sitting over a blade 1301 extending from the shaft 603. As shown in the image, after the attachment device 1201 is extended past a distal terminus of the sleeve 611, the covers 617 return to their resting position behind the extended base 1205.

FIG. 14 shows the attachment device 1201 being used to pierce or spear the graft 701 and into soft tissue 705. The surgeon uses the tool 601 to push the device 1201 into the site. Notably, the blade 1301 extends beyond the attachment device 1201 and the blade 1301, with a very sharp stainless steel point and edge, preferably pierces and spears into the graft 701 and into the tissue 705. Due to the way that the covers 617 close towards each other behind the base 1205 of the exposed attachment device 1201, pushing the delivery tool 601 in a distal (forward) direction will push the attachment device 1201 forward. In that disposition, the delivery tool can be used to push the attachment device 1201 into soft tissue. By pushing forward with the handle, the closed sleeve end pushes the attachment device forwards, while the sharpened tip of the sharp blade (preferably stainless steel) slices into the tissue and initiates entry into the tissue, carrying the attachment device through any graft or ligament and into the tissue.

FIG. 15 shows the base 1205 of the attachment device 1201 (e.g., tack, anchor, or fastener) having been speared (e.g., tacked, anchored, or fastened) well into the graft 701. Once the attachment device is holding the graft to the soft tissue, the surgeon can withdraw the delivery tip away from the graft. With the attachment device properly attached, the surgeon can place a finger on a hook or other ridge or grip of the handle of the delivery tool and use a thumb on the trigger to simultaneously apply forward pressure with the sleeve while withdrawing the delivery tool from the site. The distal tip of the sleeve 611 provide counterpressure on the base 1205 of the attachment device while the delivery tool is withdrawn from the site. The counterpressure negates any tendency for the sharp blade to try to withdraw the attachment device by friction forces. Instead, the counter pressure holds the attachment device stably in the target while the device is removed.

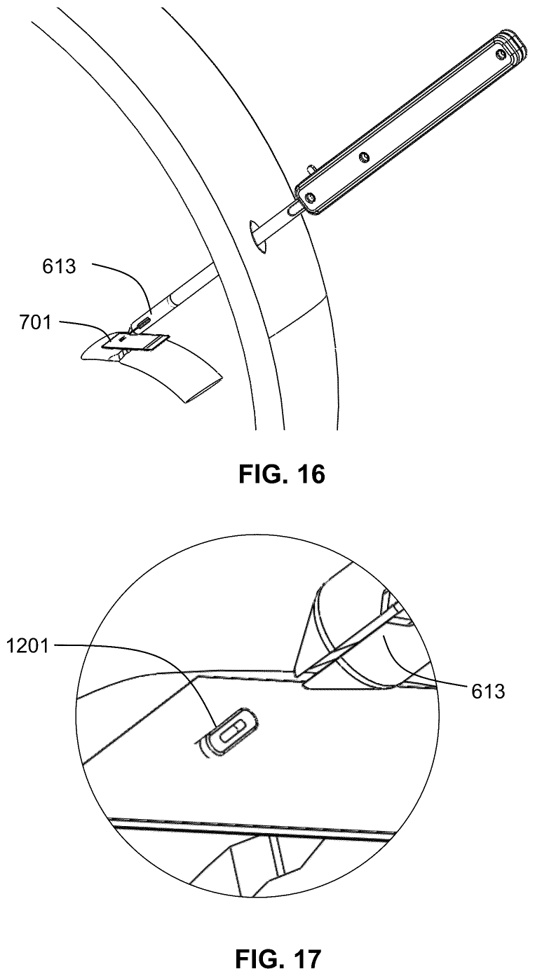

FIG. 16 illustrates the delivery tip 613 being withdrawn from the graft 701. As described, pushing the sleeve forward while gently pulling the handle back (e.g., using a thumb on the trigger and a finger on the hook) pulls the sharp blade back inside of the sleeve so that when the delivery tool is carried away from the site, removed through the incision, and potentially handled by the surgeon and attending staff, the sharp blade is fully covered by the sleeve.

FIG. 17 is a detail view of the delivery tip 613 being withdrawn from the graft 701. As shown, when the delivery tool is carried away from the site, removed through the incision, and potentially handled by the surgeon and attending staff, the sharp blade is fully covered by the sleeve.

Other embodiments are within the scope of the disclosure. Some embodiments use an attachment device with more than one leg while still operating essentially similarly to those described above.

In multi-leg embodiments, an attachment device has legs extending from a base with open portions through the base through which sharp delivery blades extend during delivery (just as described above).

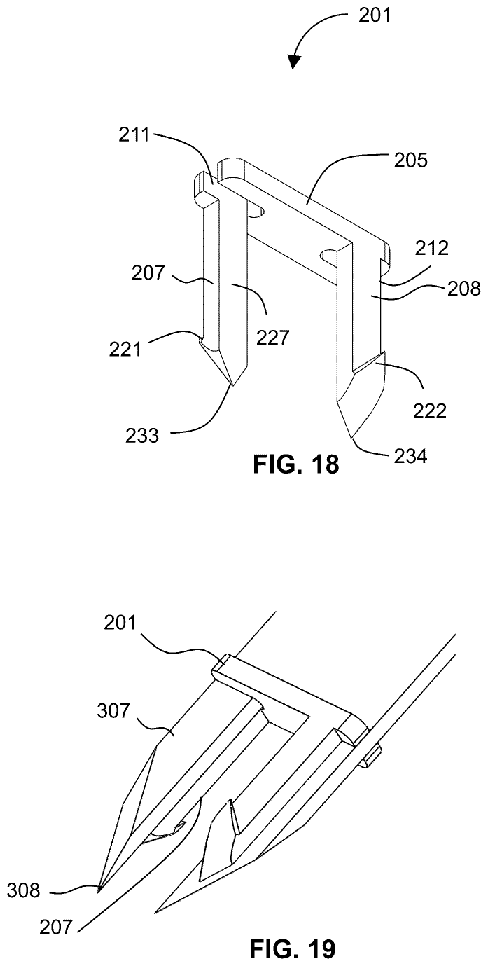

FIG. 18 shows an implantable soft-tissue attachment device 201 according to certain embodiments. As shown, the attachment device 201 includes a base 205 extending substantially perpendicular from a first leg 207. The base 205 includes an open portion 211 adjacent the proximal portion of the leg 207. The implantable soft tissue attachment device 201 preferably includes a second leg 208. The first leg 207 has a first barb 221. The second leg 208 may include a second barb 222. As shown, the legs 208, 208 extend from a proximal portion to respective distal tips 233, 234 that are shaped with a taper. The base 205 is connected to proximal portions of the legs 207, 208. The base 205 extends substantially perpendicular to the legs 207, 208.

The open portions 211, 212 allow a piercing tool to extend through the base and along a side of the leg. The legs 207, 208 comprises respective barbs 221, 222 protruding therefrom in positions that do not overhang the open portion of the base.

Importantly, the first leg 207 has at least one substantially planar surface 227 along a length of the first leg. The open portion 211 has an inward facing flat portion, wherein the substantially planar surface 227 and the flat portion define a continuous flat surface adjacent an open space that allows a flat blade to slide proximally and distally against the continuous flat surface. The barb 221 allows the first leg to be pushed in a distal direction into soft tissue and to impede the first leg from being withdrawn in a proximal direction out of the soft tissue.

As shown, the distal tip 233 terminates at a sharp point and the taper extends smoothly from the sharp point over the first barb 221 protruding from the first leg 207 in a direction that does not overhang the open portion 211 of the base (same for second barb 222). The first leg and the first open portion define a flat surface 227 along which a delivery blade 307 may slide through the base 205 and the second leg and the second open portion define a second flat surface along which a second delivery may slide through the base.

A proximal side of the base 205 comprises a pushable surface by which the first leg and the second leg may be pushed into soft tissue. The first distal tip 233 and the second distal tip 234 are sharpened to pierce the soft tissue. A distal side of the base, between the first leg and the second leg, presents a substantially flat surface operable to hold a graft or ligament against soft tissue when the first leg and the second leg are pushed into the soft tissue. Preferably the base is broader than the first leg and operable to hold a graft against tissue and wherein the open portion is an aperture through the cap.

A feature of the invention is that the barbs 221, 222 do not extend medially, in to the space between the two legs 207, 208 (e.g., towards each other). To maximize gripping power into soft tissue, the barbs may extend in opposed directions outward from the device (e.g., outward from the base 205).

Open portions 211, 212 in the base 205 allow a piercing tool to extend through the base 205 and along sides of the legs 207, 208. As shown, the first leg 207 comprises a barb 221 protruding from the first leg 207 and the barb 221 does not overhang the open portion 211 of the base. That is, importantly, the barbs 221, 222 do not block (or occlude) the open portions 211, 212. The depicted embodiment includes the second leg 208 extending from the base 205 to a second distal tip that is shaped with a second taper, the second leg being substantially parallel to the first leg.

The first leg 207 extends to a distal tip 233 that terminates at a sharp point. The taper preferably extends smoothly from the sharp point over the first barb 221 (which protrudes from the first leg 207 in a direction that does not overhang the open portion 211 of the base 205). The base 205 functions as a cap that is broader than the legs 207, 208 and operable to hold a graft against tissue

FIG. 19 shows the attachment device 201 (e.g., tack, anchor, or fastener) mounted on a sharp blade 307 of a delivery tool. As shown, the sharp blade 307 extends beyond the first leg 207 of the attachment device 201. The blade 307 can be retracted 115 from the leg 207 of the attachment device 201.

The method 101 includes piercing 107 soft tissue with a pointed tip 308 of the blade 307 that extends beyond the distal end of the leg 207 of the attachment device 201. The pointed tip of the blade pierces 107 the tissue (and the graft or ligament to be attached to the tissue). The attachment device 201 is enters the soft tissue and the base 205 acts as a cap to attach 111 the graft or ligament to the tissue. The attachment device includes: a first barb 221 protruding from the first leg 207. The first barb 221 does not block the first sharp blade 307, and a second barb 222 protrudes from the second leg 208 in a direction that does not interfere with the second blade. Preferably the first barb and the second barb extend outward from the first leg and the second leg to grip soft tissue at locations that are not medial between the first and second leg. The first leg and the first open portion define a flat surface along which the first sharp blade lies (same for second leg) allowing the disposition shown in FIG. 3.

In some embodiments, systems wherein the first leg and first sharp blade cooperate to define a tissue-piercing element terminating at a sharpened tip of the first sharp blade, and in which the second leg and the second blade cooperate to a define a second tissue-piercing element terminating a second sharpened tip of the second blade.

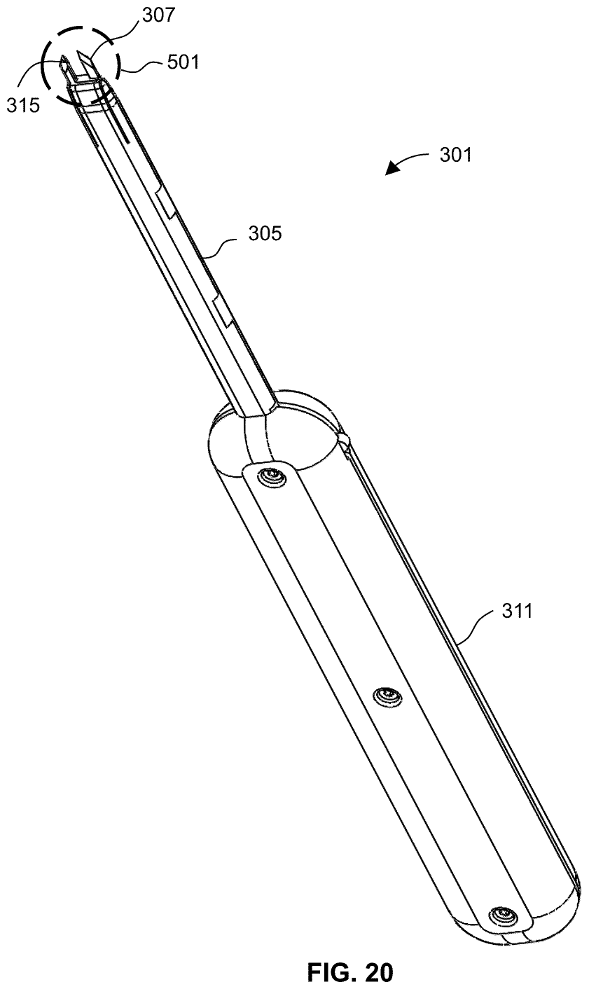

FIG. 20 shows a delivery tool 301 for delivering the soft tissue attachment device 201 into soft tissue. The delivery tool 301 includes an elongated shaft 305 extending from a handle 311 with the first sharp blade 307 extending distally from a distal end of the elongated shaft 305. The depicted embodiment also includes a second sharp blade 315 at a delivery tip 501 of the delivery tool 301.

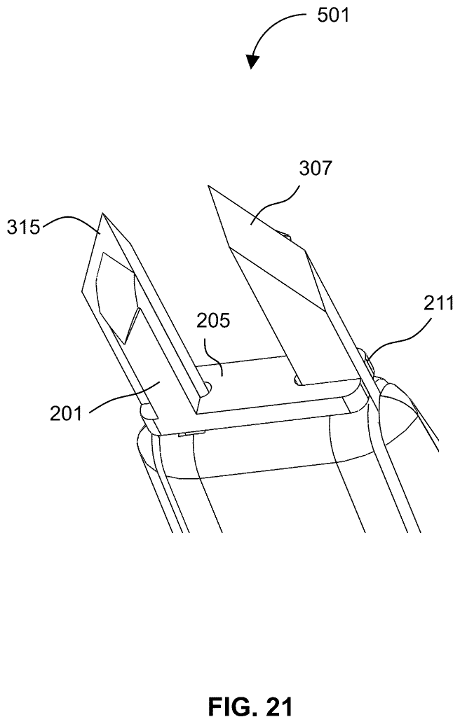

FIG. 21 is a detail view of the delivery tip 501 with the attachment device 201 (e.g., tack, anchor, or fastener) positioned on the first sharp blade 307. As shown, the first sharp blade 307 extending through the open portion 211 of the base 211 of the implantable soft tissue attachment device 201.

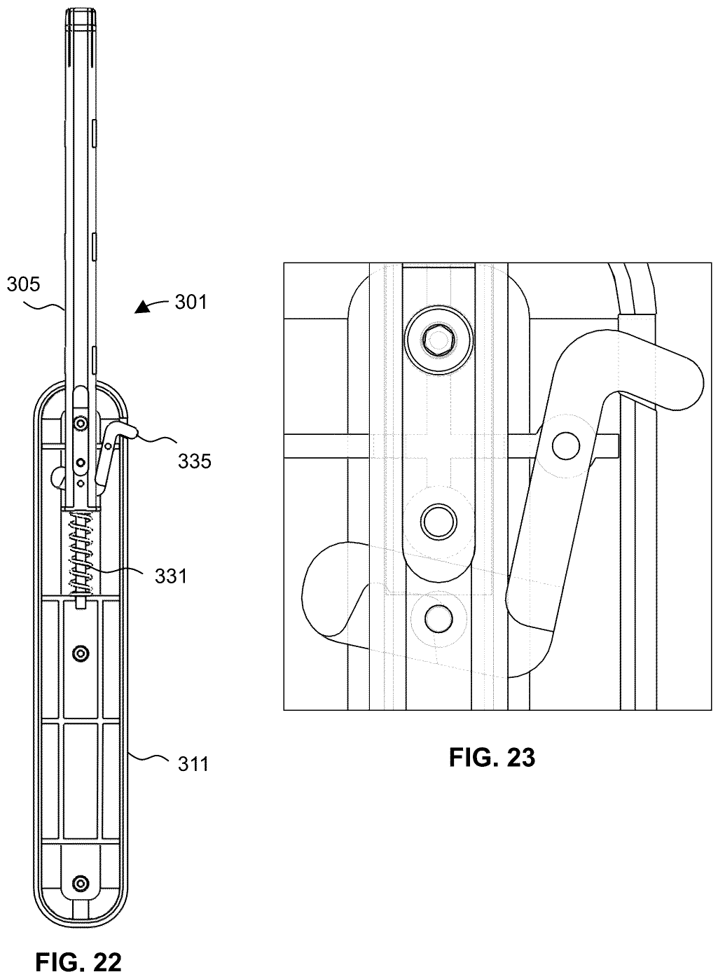

FIG. 22 shows mechanisms of the delivery tool 301 where the elongated shaft 305 extends from the handle 311. The handle 311 may include a spring biasing 331 the first sharp blade in a distal position and a switch 335 operable to restrain the first sharp blade in a proximal position.

FIG. 23 is a detail view of the switch 335. Preferably when the first sharp blade is in the proximal positioned, the first sharp blade is contained within and covered by a distal portion of the shaft. Preferred embodiments include two blades and any statement about the first blade is equally applicable to second or any subsequent blades.

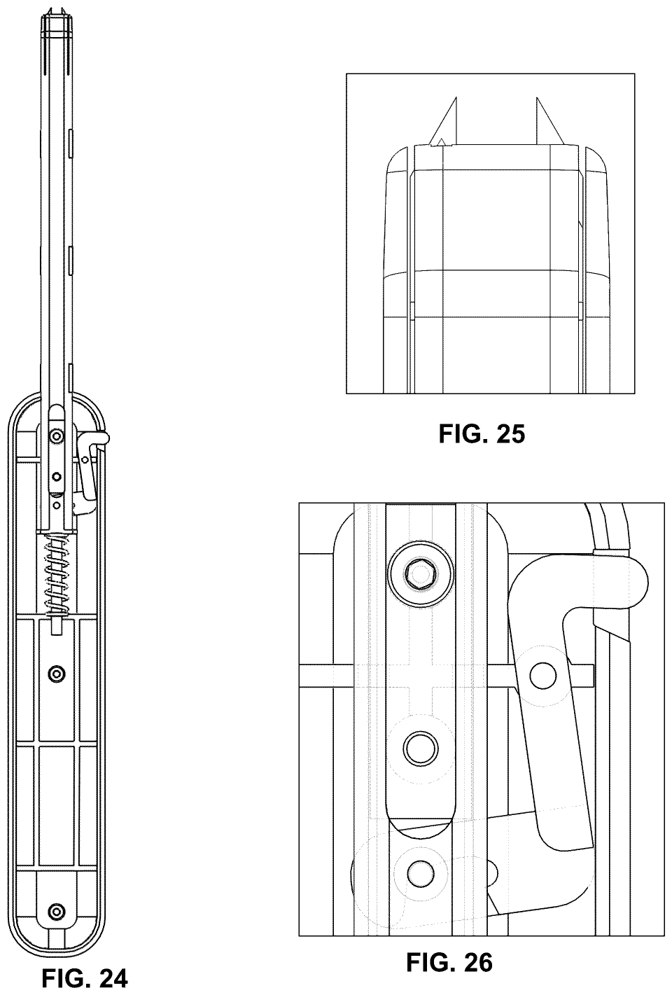

FIG. 24 shows the mechanism in the handle pushing the blades from the elongated shaft.

FIG. 25 is a detail view of the blades emerging from the shaft.

FIG. 26 shows the switch operating to release the blades.

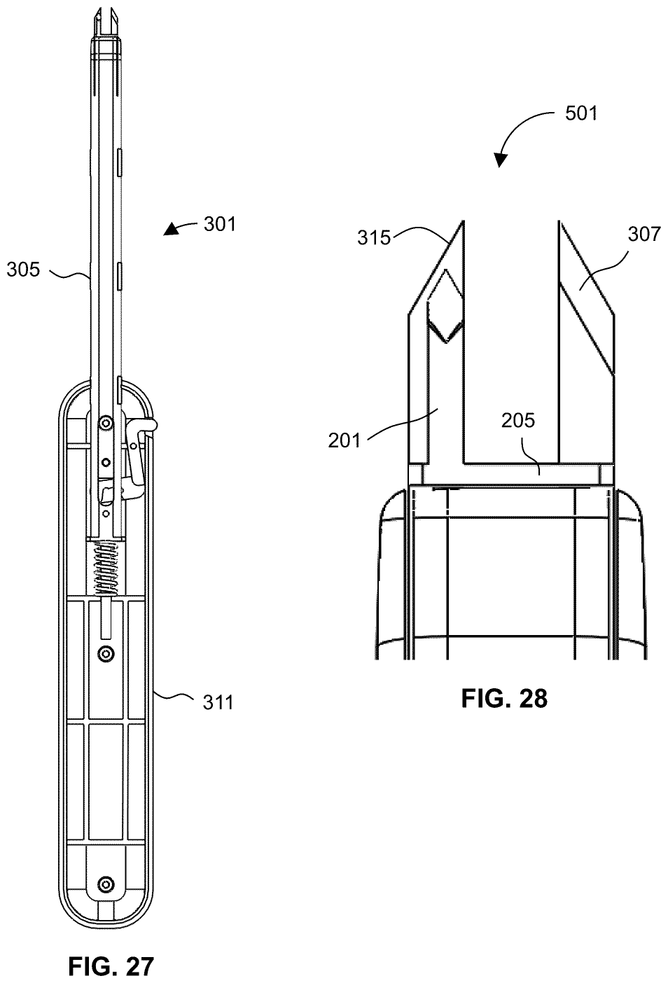

FIG. 27 shows the device 301 with the blades fully extended.

FIG. 28 is a detail view of the delivery tip 501 with the attachment device 201 positioned on the first sharp blade 307. As shown, the first sharp blade 307 extends through the open portion of the base 211 of the implantable soft tissue attachment device 201. The device 201 is part of a system for fixing soft tissue. The system includes the delivery tool 301 with the elongated shaft 305 extending from a handle 311. At least a first sharp blade 307 extends distally from a distal end of the elongated shaft 305. The attachment device 201 (e.g., tack, anchor, or fastener) is positioned on the first sharp blade, with first sharp blade 307 extending through the open portion of the base 205.

As shown, the first sharp blade 307 comprises a substantially flat piece with opposed first and second surfaces extending distally to a sharpened piercing tip. Preferably, the sharpened piercing tip of the first sharp blade 307 extends distally beyond the distal tip of first leg of the attachment device 201. The first leg of the attachment device lies flat against a surface along a side of the first sharp blade (FIG. 5 also shows this well).

When the first sharp blade is pushed distally into soft tissue, a tip of the first sharp blade pierces a hole into the tissue and the distal tip of the first leg is pushed into the hole. After the first sharp blade is pushed into tissue to carry the attachment device into the tissue, the first sharp blade can be retracted into the shaft (FIG. 6), withdrawing the first sharp blade proximally through the open portion of the base of the attachment device. When the blade is withdrawn proximally, one or more barbs 221, 222 on the attachment device hold the attachment device in the tissue. When the attachment device 201 is mounted on the delivery tool 301, the first blade presents a first sharpened point extending distally from the distal tip of the first leg, and the second blade presents a second sharpened point extending distally from a second distal tip of the second leg.

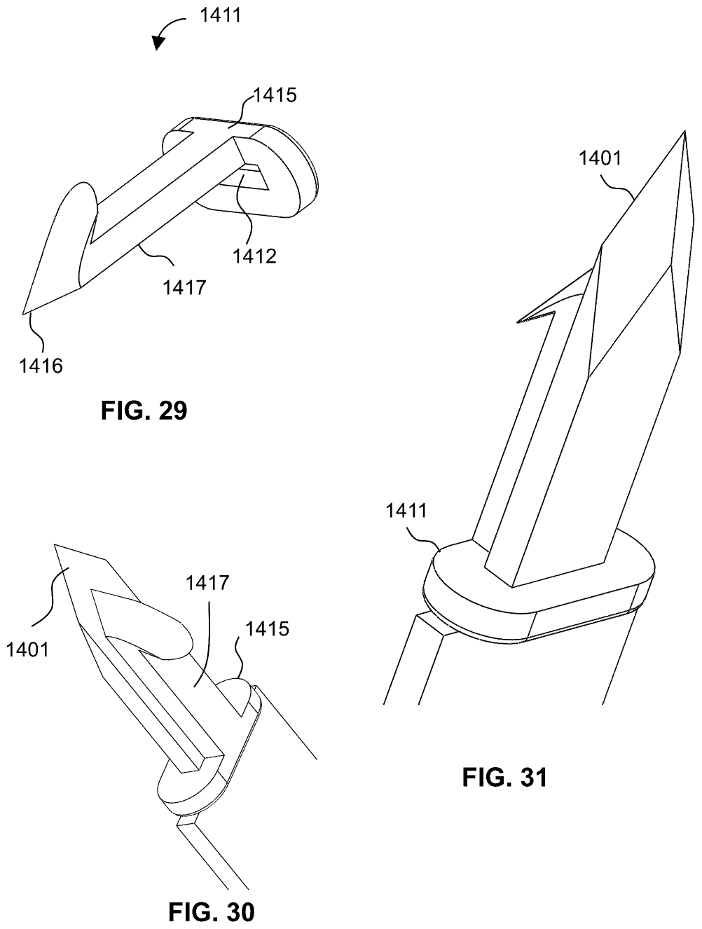

FIG. 29 shows another single leg embodiment of a soft tissue attachment device 1411 (e.g., tack, anchor, or fastener). The attachment device 1411 includes at least a first leg 1417 extending from a proximal portion to a distal tip 1315 that is shaped with a taper and a base 1415 connected to the proximal portion of the first leg 1417. The base 1415 extends substantially perpendicular to the first leg 1417. The base 1415 comprises an open portion 1412 adjacent the proximal portion of the leg.

FIG. 30 shows a piercing tool 1401 extended through the base and 1415 along a side of the leg 1307.

FIG. 31 shows the single leg embodiment of a soft tissue attachment device 1411 on the piercing tool 1401 from another perspective.

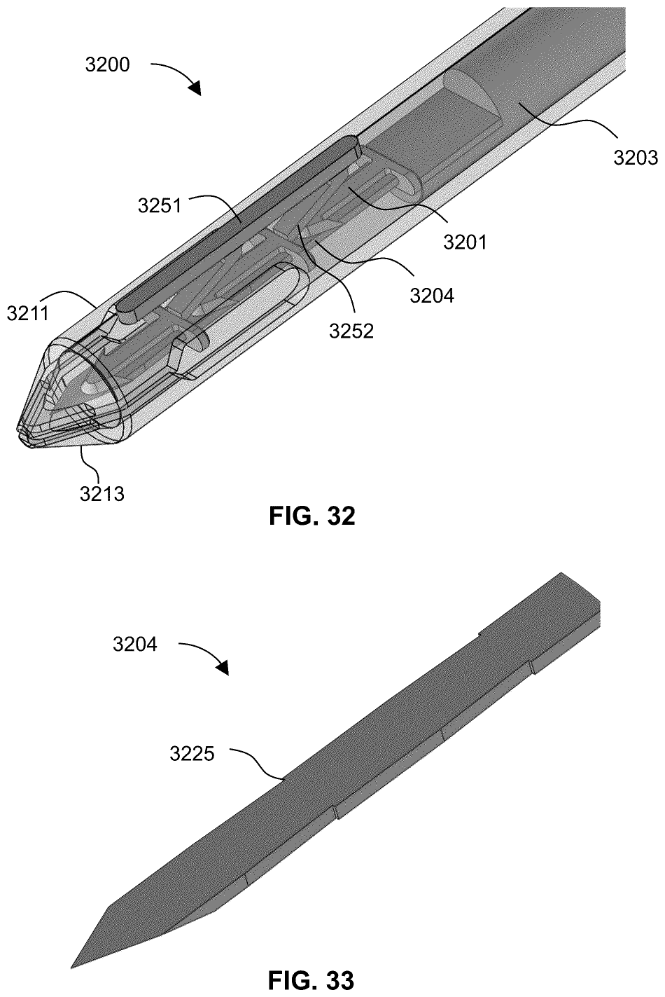

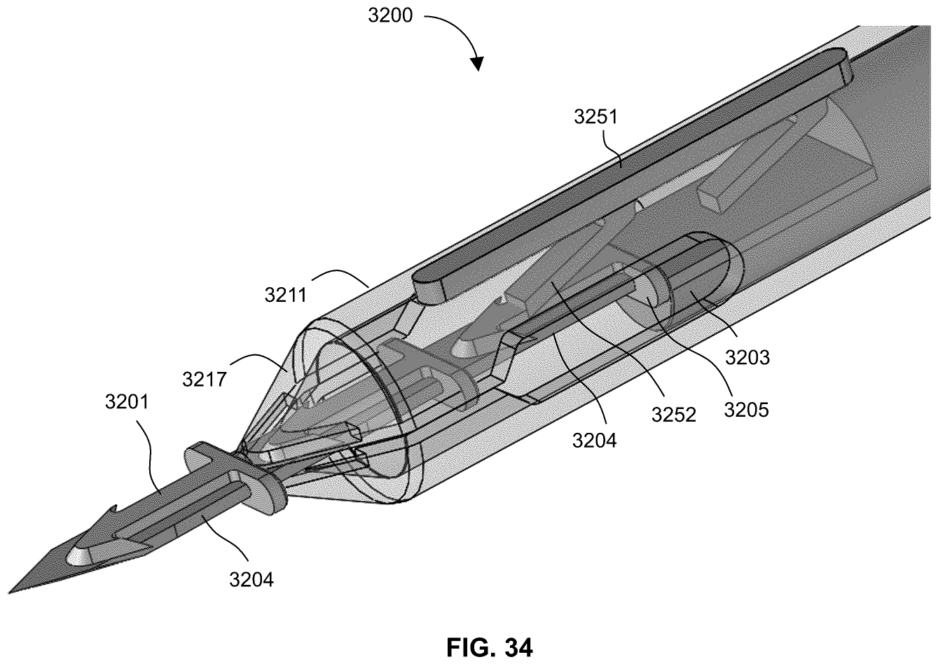

FIG. 32, FIG. 33, and FIG. 34 illustrate an embodiment of a magazine mechanism that allows the application of multiple fasteners using a single device

FIG. 32 shows a distal tip of a multi-anchor delivery tool 3200 for delivering a plurality of implantable anchors 3201. The delivery tool 3200 includes a handle (not shown here, same as handle in FIG. 10) with a shaft 3203 extending therefrom with a blade 3204 extending from the shaft 3203. The shaft is covered by a sleeve 3211. The sleeve 3211 extends distally to a sloped delivery tip 3213. The sleeve 3211 is preferably slidably disposed over the elongated shaft 3203 and blade 3204. The sleeve 3211 can be translated (relative to the handle, shaft, and delivery tip) between a proximal and distal position. Multiple fasteners 3201, carried on the blade 3204, may be delivered using the single device 3200. According to this embodiment the blade 3204 holds several fasteners 3201 along it length. The device 3200 includes a comb 3251

The comb 3251 has a series of flexible teeth 3252 and is located on the sleeve 3211 and above the fasteners 3201. When the sleeve 3211 is retracted back, the teeth 3252 will be located behind the fasteners 3201 such that when the sleeve 3211 is advanced forward (during the retraction of the blade), a new fastener gets positioned at the distal end of the blade, ready for the next application.

FIG. 33 shows the blade 3204 for an embodiment of a delivery tool 3200 that can carry a plurality of implantable anchors. The blade 3204 may include a series of sloped steps 3225 at the lateral edges of the blade 3204, which prevent the fasteners from being pulled back (along the blade 3204) during the sliding of the comb 3251 backwards, which draws the tips of the teeth 3252 over the loaded fasteners 3201.

FIG. 34 shows this distal end of the device 3200. The distal-most fastener 3201 is inserted into tissue by pushing forward with the handle of the device 3200. In this embodiment, the conical tip 3217 assists in pushing the distal-most fastener 3201 into the tissue. Once the distal-most fastener 3201 is in tissue, the conical tip can be pressured forward, applying counter pressure to the fastener while the blade 3204 is withdrawn.

As the blade 3204 is withdrawn from the implanted fastener, the teeth 3252 of the comb 3251 deform and return as they pass over the bases 3205 of the fasteners 3201 carried within the sleeve 3211. Note that the sleeve 3211 and comb 3251 are once first piece, while the handle, shaft 3203, and blade 3204 are preferably one second piece. Preferable the only moving parts of the delivery tool 3200 are the first piece and its ability to translate relative to the second piece. After delivery, when the sleeve 3211 is retracted, the teeth 3252 land behind the bases 3205 of the enclosed (by sleeve 3211) fasteners 3201. Then, when the sleeve 3211 is slide in a distal direction, the teeth 3252 carry a new distal most fastener 3201 out from a position deep on the blade 3204 onto the delivery tip of the blade, reforming the version shown in FIG. 32 (albeit with one fewer of the fasteners 3201 loaded in the magazine).

Due to the nature of interactions among the rigid material (e.g., stainless steel) of the blade 3204 and the more pliable fasteners (e.g., bioabsorbable polymer) 3201, the steps 3225 prevent that fasteners from being dragged back by the comb 3251 while the sleeve is being retracted.

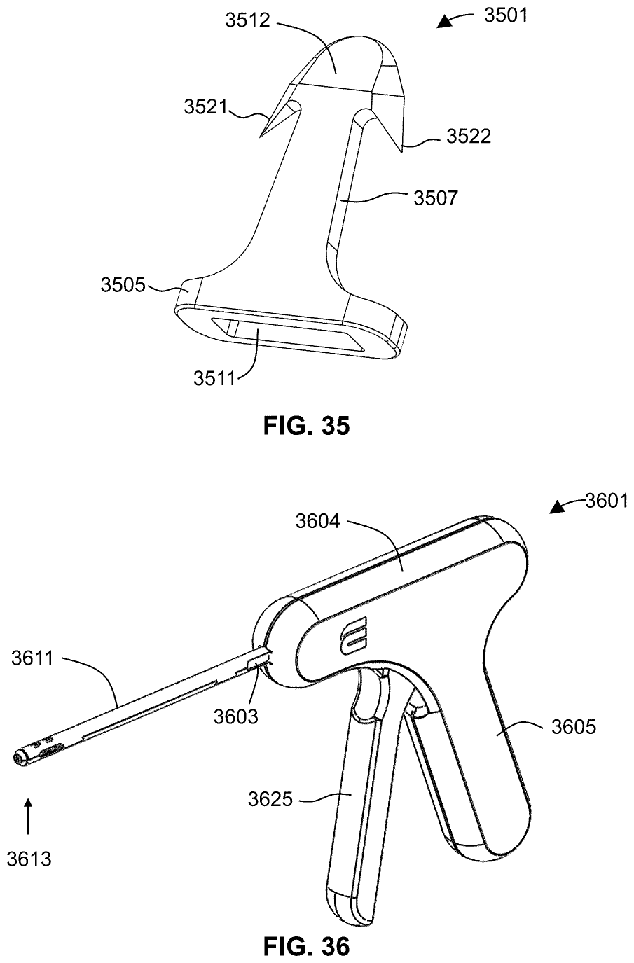

FIG. 35 shows a tapered and shaped version of an attachment device 3501 (aka tack, anchor, or fastener) that is useful for attaching or fastening tendon, ligament, or other soft material together, e.g., attaching a tendon to soft tissue. The tapered attachment device 3501 has a tapered leg 3507 extending from a proximal portion to a distal tip, in which the tapered leg 3507 is shaped with a taper, the distal tip is tapered and rounded and has a sloped face 3512.

A base 3505 is connected to the proximal portion of the tapered leg 3507. The base 3505 extends substantially perpendicular to the tapered leg 3507. The base 3505 comprises an open portion 3511 adjacent the proximal portion of the tapered leg 3507. The open portion 3511 allows a piercing tool with a blade to extend through the base 3505 and along a side of the tapered leg 3507. The attachment device may be made of an absorbable material, a bioabsorbable material, or a biological material. The attachment device 3501 may be made of an absorbable polymer (e.g. PEEK, POM, Nylon, HDPE), bioabsorbable polymer (e.g. PLA, PGA, PDS, PLGA), or a biological metrical such as collagen or bone.

As shown, the tapered leg 3507 comprises at least one barb 3521 protruding therefrom, preferably a pair of barbs 3521, 3522 extending in opposed lateral directions from each other. The tapered leg 3507 has at least one substantially planar surface along a length of the leg. The open portion 3511 has an inward facing flat portion, such that the substantially planar surface and the flat portion define a continuous flat surface adjacent an open space that allows a flat blade to slide proximally and distally against the continuous flat surface. The barbs 3521, 3522 allow the tapered leg 3507 to be pushed in a distal direction into soft tissue and to impede the first leg from being withdrawn in a proximal direction out of the soft tissue. Preferably, the barbs 3521, 3522 do not extend into the open space through which the flat blade may slide proximally and distally against the continuous flat surface. The attachment device 2501 may include at least on perpendicular barb on a side of the leg obverse to the planar surface.

The sloped face 3512 may be provided to reduce the penetration loads at the front section of the attachment device 3501, which functions as a tack. As shown, the tapered leg 3507 is wider toward the base 3505. Under conditions that could lead to pullout of the attachment device 3501 from the tissue, the proximal portion of the tapered leg 3507 is subjected to higher deformation and loads than other portions of the attachment device 3501. Each of the sloped face 3512 and the tapered leg 3507 both alone and in combination have been found to promote ease of surgical delivery and durable attachment once delivered e.g., when anchoring a tendon to sot tissue.

FIG. 36 shows a “pistol grip” version of a tendon or soft tissue pistol-grip delivery tool 3601 for delivering a soft-tissue fastener such as the surgical attachment device 1201 or tapered attachment device 3501. The pistol grip” soft tissue pistol-grip delivery tool 3601includes a body 3604 with a handle 3605 and a shaft 3603 extending therefrom. The shaft 3603 is covered by a sleeve 3611. The sleeve 3611 extends distally to a delivery tip 3613. Inside the delivery tip, the shaft 3603 terminates with a sharp blade. The sleeve 3611 is preferably slidably disposed over the elongated shaft 3603. The sleeve 3611 can be translated (relative to the shaft 3603) between a proximal and distal position. The delivery tool 3601 includes a trigger 3625 extending from a portion of the body 3604. A surgeon can translate the sleeve between proximal and distal dispositions by operating the trigger 3625.

The depicted ‘pistol’ grip handle 3605 may operate via a crank shaft mechanism that moves the sleeve 3611 back and forth. The depicted “pistol grip” mechanism has been found to be convenient to use and ensures that the sleeve 3611 will not move forward prematurely.

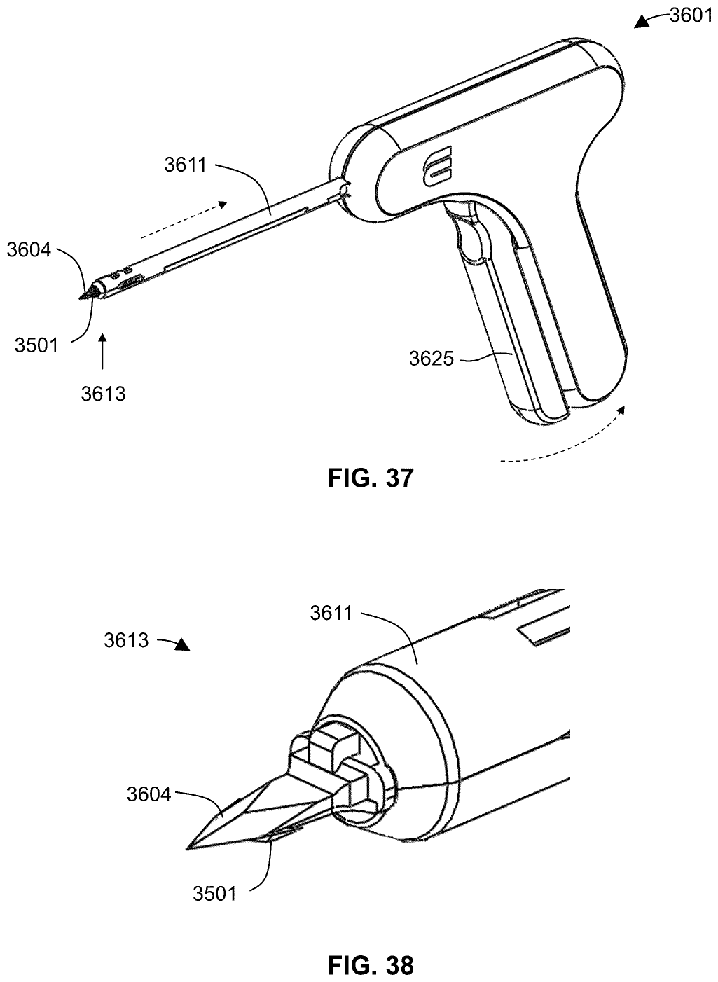

FIG. 37 illustrates a first step in operation of the “pistol grip” version of the pistol-grip delivery tool 3601. A user squeezes the trigger 3625 to thereby pull back the sleeve 3611, exposing the tip of the blade 3604 and the tack or attachment device 3501.

FIG. 38 is a close-up of the delivery tip 3613 of the delivery tool 3601, showing the sharpened blade 3604 extending forward from the sleeve 3611 with an attachment device 2501 held thereto.

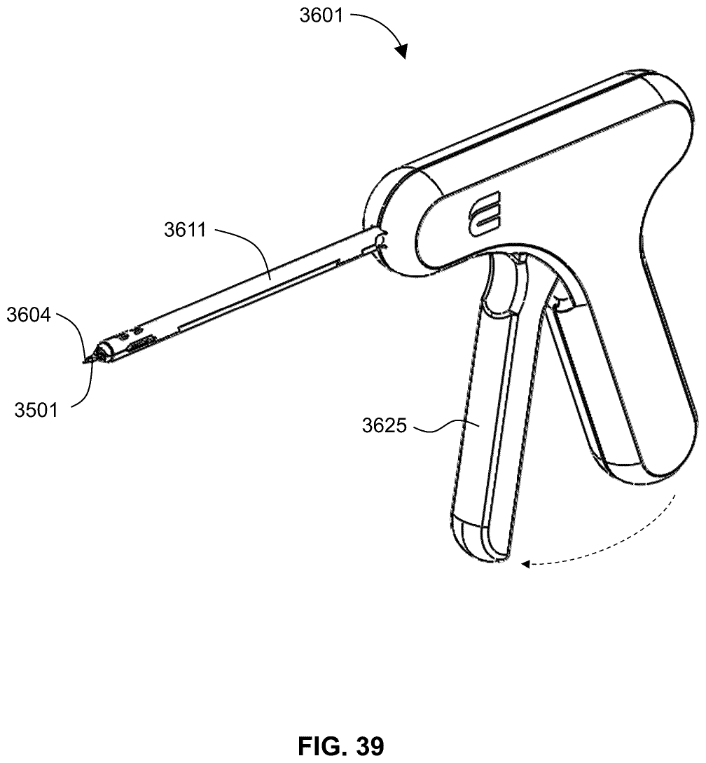

FIG. 39 shows that once the trigger 3625 is released, it returns to its initial position (by an internal spring), leaving the sleeve 3611 retracted. The configuration and stage of operation shown here is the configuration in which a surgeon uses the device 3601 to deliver an attachment device 3501 into soft tissue.

Once the attachment device 3501 is inserted into the soft tissue, the user of the device may squeeze the trigger 3625 a second time to complete the delivery.

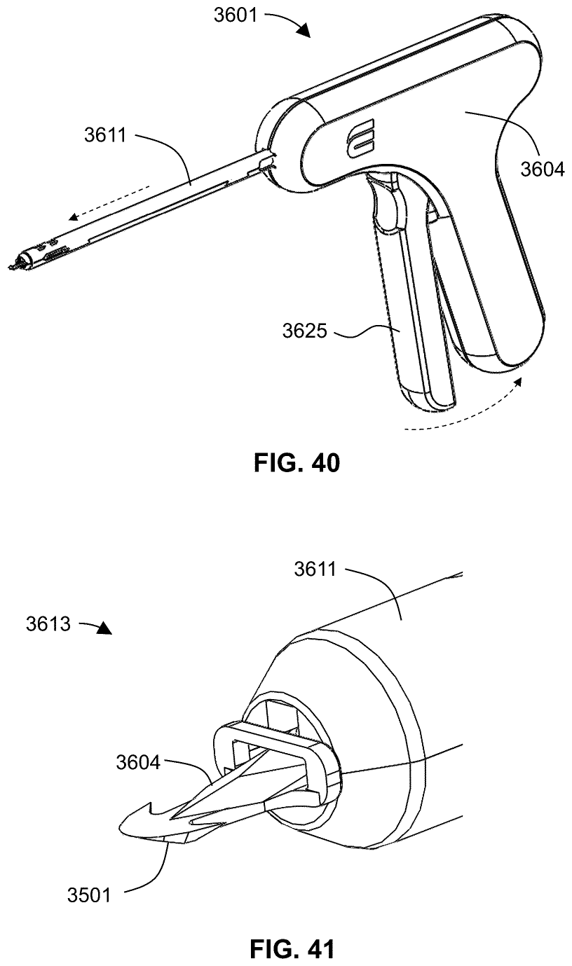

FIG. 40 illustrates that a second pull (compression) of the trigger 3625 advances the sleeve 3611 back in a forward, or distal, direction from the device 3601. The depicted pull of the trigger 3625, noting that the attachment device 2501 has been inserted into the tissue (not pictured) pushes the sleeve 3611 forward, sliding the attachment device 3501 forward off of the blade 3604, while also advancing forward second and subsequent tacks loaded within the sleeve 3611 of the delivery tool 3601.

FIG. 41 gives a detail view of the delivery tip 3613 during the second trigger pull, while the sleeve 3611 is pushing the attachment device 3501 forward off of the blade 3604.

The depicted mechanical operations rely on ratcheting geared crank mechanisms inside of the body 3604 of the pistol-grip delivery tool 3601, which mechanisms will now be shown and discussed.

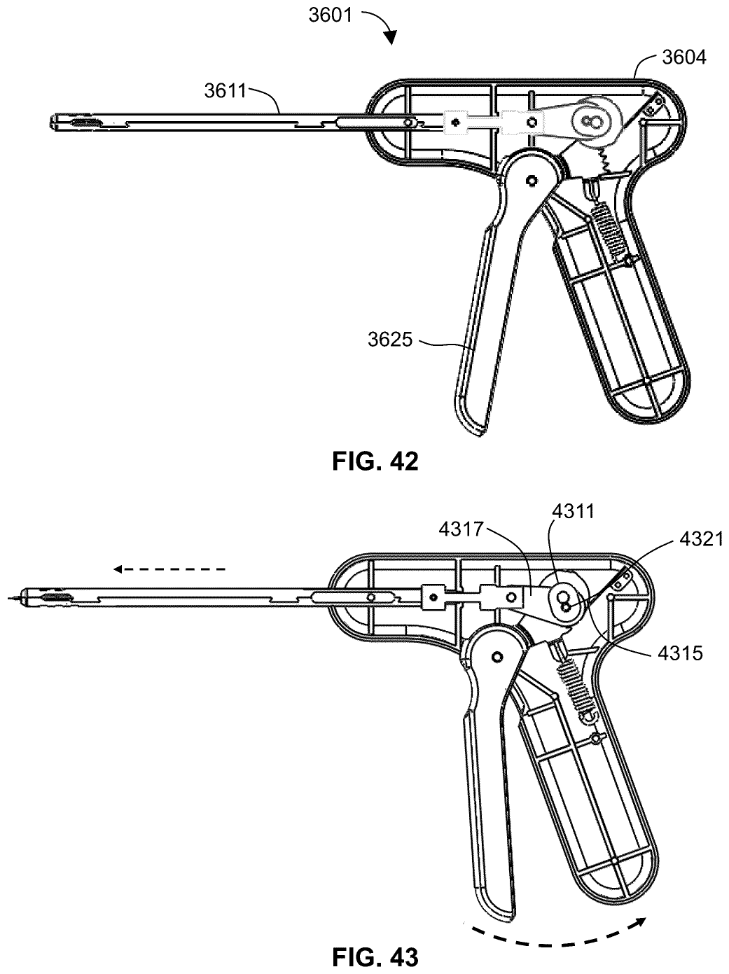

FIG. 42 is a cutaway view from a first side of the delivery tool 3601 showing details of the geared crank mechanisms inside of the body 3604 connecting the sleeve 3611 to the trigger 3625.

FIG. 43 shows the displacement of shaft 3603 when trigger 3625 is squeezed. One complete squeeze of the trigger 3625 causes a crank shaft hub 4311 to rotate 180 degrees. A return limiter 4315 prevents rotation of the crank shaft hub 4311 in an opposite direction. The sleeve 3611 is connected to the crank shaft hub 4311 via a connecting rod 4317 that is attached to the crank shaft hub at a pivot point 4321 that is not at the rotational center the crank shaft hub 4311. Thus, when the crank shaft hub 4311 rotates, the sleeve 3611 translates reciprocally (on the mechanical principle by which pistons are driven by a rotating crank shaft).

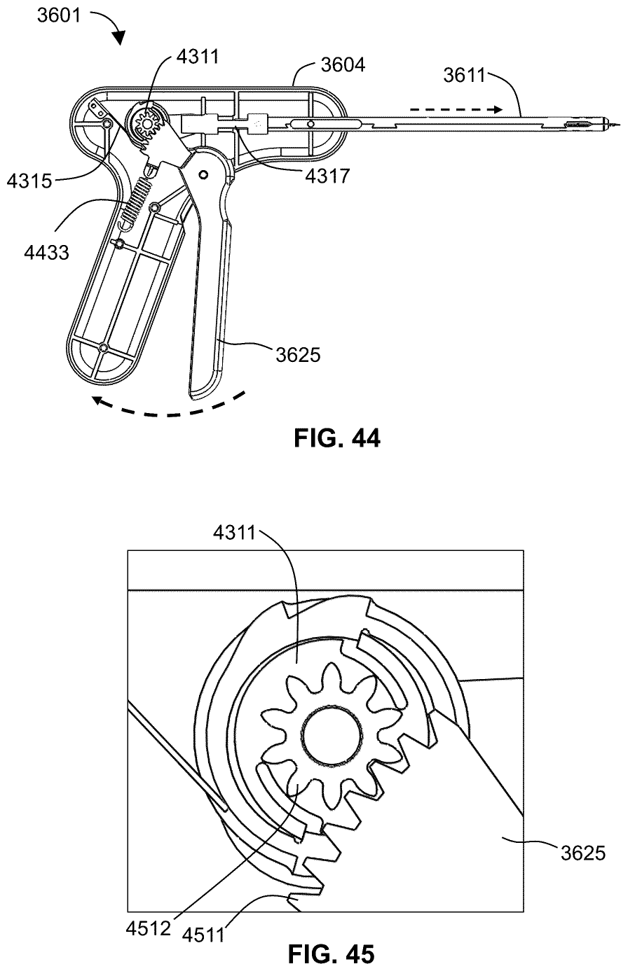

FIG. 44 is a cutaway view from a second side of the delivery tool 3601 showing a crank shaft hub 4311 to which the connecting rod 4317 is pivotably attached. The return limiter 4315 prevents rotation of the crank shaft hub 4311 in an opposite direction. The sleeve 3611 is connected to the connecting rod 4317. The trigger 3625 is connected via a trigger gear to a ratchet gear.

FIG. 45 is a detail view showing the trigger gear 4511 engaged with the ratchet gear 4512. The ratchet gear 4512 is housed inside the crank shaft hub 4311, implementing a one direction ratchet mechanism. A full stroke of the trigger 3625 generate a 180 degrees rotation of the of the ratchet gear 4512 together with the crank shaft hub 4311. Once released, the trigger 3625 returns to its initial position by virtue of the return spring 4433. Because the return spring 4422 moves the trigger gear 4511, the ratchet gear 4512 turns in a reverse direction by 180 degrees. However, the crank shaft hub 4311 dose not rotate during this motion and is held by the return limiter 4315.

In preferred embodiments, a delivery tool 601, 3601 of the disclosure may be pre-loaded with multiple attachment devices (e.g., tacks, anchors, or fasteners).

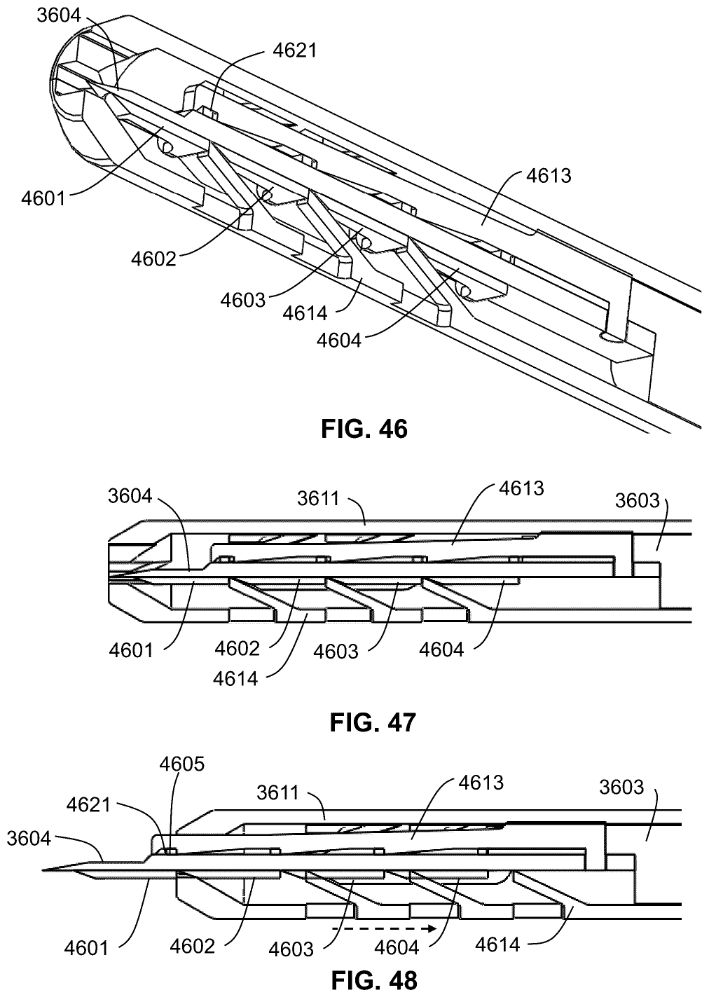

FIG. 46 is a perspective cutaway view of a delivery tip of a delivery tool of the disclosure. A first attachment device 4601 (e.g., tack, anchor, or fastener), a second attachment device 4602, a third attachment device 4603, and a fourth attachment device 4604 are loaded therein. The device includes a static comb 4613 and a dynamic comb 4614 that cooperate to coordinate the delivery of one attachment device at a time.

FIG. 47 is a cutaway side view through the delivery tip, showing the sleeve 3611 covering the first attachment device 4601 (and subsequent) and the blade 3604. The static comb 4613 is fixed with respect to the shaft 3603. The dynamic comb 4614 is fixed with respect to the sleeve 3611.

FIG. 48 shows the blade 3604 and the first attachment device 4601 being uncovered by the static comb 4613 being pulled distally with the sleeve 3611. The shaft 3603 and the dynamic comb 4614 are advanced in a distal direction (relative to the sleeve 3611). The depicted device provides a mechanism of tack advancing during operation. The static comb 4613, which includes a series of sloped steps, is connected to the central shaft 3603. This comb 4613 is designed the prevent backward movement of the tacks 4601 once the sleeve 3611 is retracted back. Once the sleeve 3611 is pushed forward, the dynamic comb 4614 pushes the tacks 4601 forwarded while the static comb 4613 is push upward.

A notch 4621 at the distal end of the static comb 4613 is designed to prevent unintentional release of the fastener prior to insertion. Each of the first attachment device 4601, the second attachment device 4602, the third attachment device 4603, and the fourth attachment device 4604 has a base 4605 that surrounds the blade 3604 and sits within the notch 4621 when the attachment device is in the distal-most ready position.

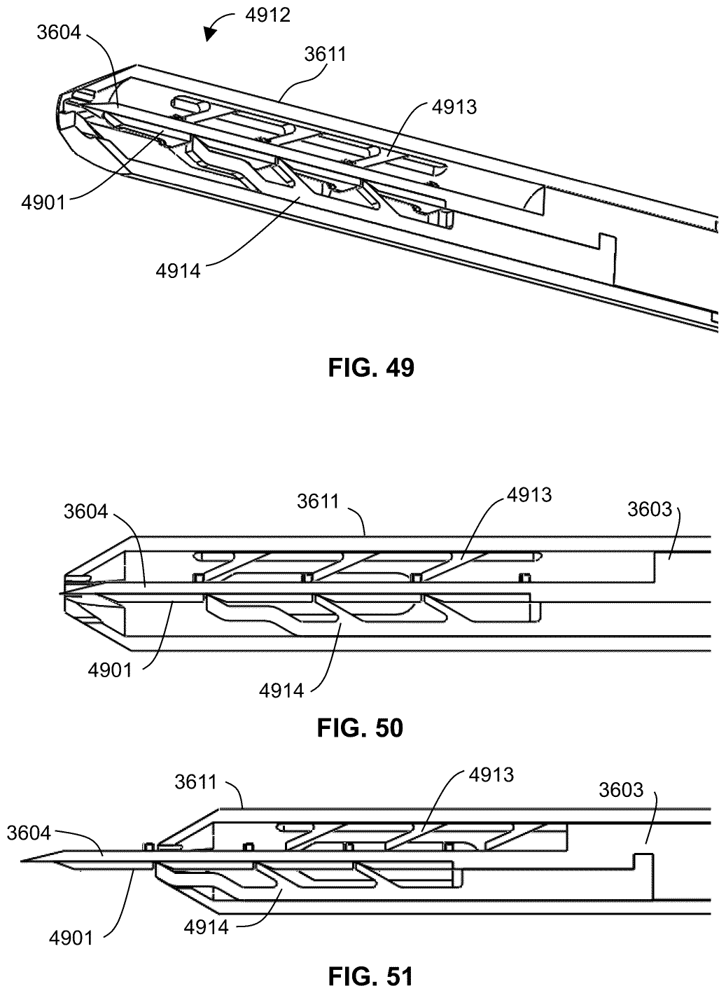

FIG. 49 is a perspective cutaway view of a delivery tip 4912 that may be included on any delivery tool of the disclosure. A first attachment device 4901 (e.g., tack, anchor, or fastener) and others are loaded therein. The device includes a static comb 4913 and a dynamic comb 4914 that cooperate to coordinate the delivery of one attachment device at a time. As shown, the static comb 4913 and the dynamic comb 4914 form part of a one-at-a-time delivery mechanism disposed within sleeve 3611. In the depicted embodiment, the static comb 4913 has individual teeth that are free to flex independently from each other. The static comb 4913 is static in that it has a fixed lateral position with respect to the shaft 3603.

FIG. 50 is a cutaway side view through the delivery tip 4912, showing the sleeve 3611 covering the first attachment device 4901 (and subsequent) and the blade 3604. The static comb 4913 is fixed with respect to the shaft 3603. The dynamic comb 4914 is fixed with respect to the sleeve 3611. Each tooth of the static comb 4913 is free to independently deform away from a center axis of the sleeve 3611 allowing the attachment device(s) to slide past. As shown, the tips of the teeth of the static comb 4913 may each sit behind a lip or base on a respective attachment device 4901 to prevent back-motion of that attachment device.

FIG. 51 shows the delivery tip 4912 with the blade 3604 and the first attachment device 4601 being uncovered by the static comb 4913 being pulled distally with respect to the sleeve 3611. The shaft 3603 and the dynamic comb 4914 are advanced in a distal direction (relative to the sleeve 3611). The depicted device provides a mechanism of tack advancing during operation. The static comb 4913, which includes a series of sloped steps, is connected to the central shaft 3603. This comb 4913 is designed the prevent backward movement of the tacks 4601 once the sleeve 3611 is retracted back. Once the sleeve 3611 is pushed forward, the dynamic comb 4914 pushes the tacks 4601 forwarded while the static comb 4913 is push upward.

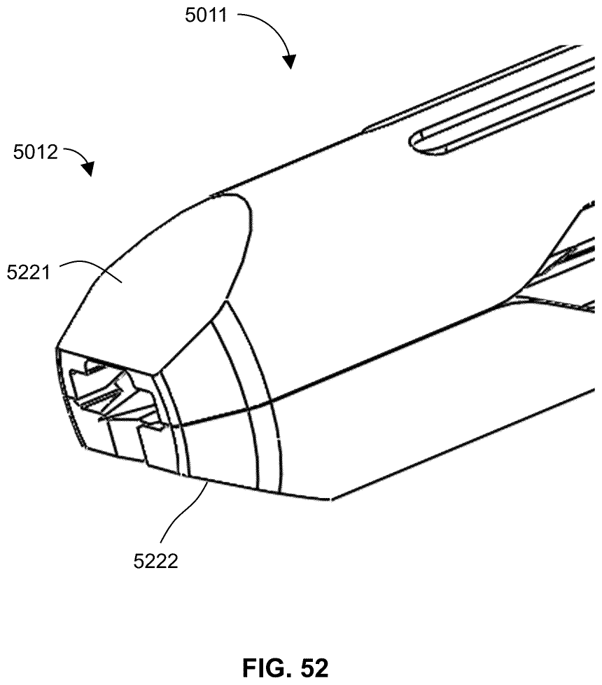

FIG. 52 shows a tapered insertion sleeve 5011 that may be used with any delivery tool of the disclosure. When a delivery tool includes the tapered insertion sleeve 5011, a deliver tip 5012 of the delivery tool has tapered, sloped faces 5221, 5222. Due to the sloped faces 5221, 5222, the delivery tip 5012 has a profile that is tapered at the distal end, reducing contact with any graft, cannula, or surrounding tissue, gently guiding the delivery tool to the site, and allowing better access and penetration in shallow angles of approach. As shown, the delivery tool comprises a tapered insertion sleeve 5011 disposed over the shaft. The tapered insertion sleeve 5011 is slidable between a proximal and a distal position. When the tapered insertion sleeve 5011 is in the distal position, the tapered insertion sleeve 5011 covers the first sharp blade 3604. The tapered insertion sleeve 5011 has the sloped faces 5221, 5222 and a slit that divides the delivery tip 5012 into two half covers that can bend away from each other.

In use, when an attachment device 4601 is positioned on the blade 3604, the tapered insertion sleeve 5011 can be retracted, by a trigger on the handle, to allow the attachment device and blade to extend beyond the tip 5012, which closes behind a base of the attachment device and may be used to push the attachment device 4601 and blade into tissue. The tapered insertion sleeve 5011 protects tissue and any cannula from the sharp blade during insertion of the delivery tool into a surgical site; the tapered insertion sleeve 5011 facilitates smooth and easy insertion through the incision.

All of the soft tissue embodiments shown herein a common feature that a delivery tool carries an attachment device (e.g., tack, anchor, or fastener) and pierces a graft or ligament and tissue, making a hole, while carrying the attachment device (e.g., tack, anchor, or fastener) into the hole and leaving the anchor in the hole when the tool is retracted. These embodiments offer reliable and easy-to-use tools for arthroscopic and orthopedic surgery. Notably, tools and methods of the disclosure do not require a first tool and a second tool to attach a tendon or similar soft tissue to an attachment point in target sot tissue. For example, some prior approaches require a first tool to begin making an attachment and then a second tool to cut and/or to tie a knot or to otherwise finalize the operation. Those approaches were suboptimal in keyhole surgery because small disturbances to the site could lose alignment, making it very difficult to place the anchor correctly. With devices, methods, and systems shown herein, a single delivery tool simultaneously cuts open the tissue, making the hole, while bringing the implantable anchor into the hole. The blade on the delivery tool does the cutting and carries in a barbed attachment device (e.g., tack, anchor, or fastener) that will stay durably attached when the delivery tool is removed from the site.

1. A surgical attachment device comprising:
a leg extending from a proximal portion to a distal tip that is shaped with a taper; and
a base connected to the proximal portion of the leg, the base extending substantially perpendicular to the leg, wherein the base comprises an open portion adjacent the proximal portion of the leg, the open portion configured to allow a sharp blade to extend through the base and along a side of the leg.

2. The device of claim 1, wherein the leg comprises a barb protruding from the first leg, wherein the barb does not overhang the open portion of the base.

3. The device of claim 2, further comprising a pair of barbs extending in opposed lateral directions from each other.

4. The device of claim 1, wherein the leg has at least one substantially planar surface along a length of the leg, wherein the open portion has an inward facing flat portion, wherein the substantially planar surface and the flat portion define a continuous flat surface adjacent an open space that allows a flat blade to slide proximally and distally against the continuous flat surface.

5. The device of claim 4, further comprising one or more barbs configured to allow the first leg to be pushed in a distal direction into soft tissue and to impede the first leg from being withdrawn in a proximal direction out of the soft tissue.

6-15. (canceled)

16. A system for fixing soft tissue, the system comprising: a delivery tool with an elongated shaft extending from a handle; at least a first sharp blade extending distally from a distal end of the elongated shaft; the attachment device of claim 1 positioned on the first sharp blade, with first sharp blade extending through the open portion of the base.

17. The system of claim 16, wherein the blade, shaft, and handle are attached together as a subunit with no moving parts, the delivery tool further comprising a sleeve slidably disposed over the shaft, wherein the sleeve comprises a conical tip and a slit into the conical tip, dividing the conical tip into two half covers that can bend away from each other.

18. The system of claim 17, wherein translating the sleeve to a proximal position on the delivery tool causes the conical tip to open as the conical tip passes over the base of the attachment device and the close behind the base, wherein after the shaft has been used to drive the attachment device through a graft and into soft tissue, the closed conical tip sitting behind the base can be used to apply counterpressure to the base while the blade withdrawn from the attachment device.

19-23. (canceled)

24. The system of claim 16, wherein the delivery tool comprises a sleeve disposed over the shaft, wherein the sleeve is slidable between a proximal and a distal position, wherein when the sleeve is in the distal position, the sleeve covers the first sharp blade.

25. The system of claim 24, wherein the sleeve has a conical tip.

26-33. (canceled)

34. A method of attaching a graft or ligament to soft tissue, the method comprising: navigating the conical tip of a system of claim 25 through an incision; retracting the sleeve to exposes the blade carrying the attachment device; piercing a graft and the soft tissue with the first sharp blade and by that carrying the attachment device through the graft and into the tissue; sliding the sleeve in a distal direction relative to the handle so that the conical tip will provide counter pressure to the base of the attachment device while the blade is being pulled out from the tissue by pulling the handle back. removing the delivery tool from the incision.

35-40. (canceled)

41. A system for fixing soft tissue, the system comprising:
a delivery tool with an elongated shaft extending from a body;
a handle and a trigger extending from the body; a sharp blade extending distally from a distal end of the elongated shaft;
an attachment device disposed on the blade, the attachment device comprising a tapered leg extending from a proximal portion to a distal tip, and a base connected to the proximal portion of the leg, the base extending substantially perpendicular to the tapered leg, wherein the base comprises an open portion adjacent the proximal portion of the leg, wherein the sharp blade extends through the base and along a side of the tapered leg when the attachment device is positioned on the sharp blade; and
a sleeve extending from the body and covering the sharp blade and the attachment device, wherein squeezing the trigger towards the handle pulls the sleeve in a proximal direction towards the handle, exposing the sharp blade and the attachment device.

42. The system of claim 41, wherein the trigger comprises gears engaged with a crank shaft hub that is connected to the sleeve via a connecting rod, whereby squeezing the trigger rotates the crank shaft hub, pulling the sleeve via the connecting rod to translate over, and uncover, the sharp blade.

43. The system of claim 42, wherein the crank shaft hub is engaged with a ratchet mechanism, and wherein squeezing the trigger a second time pushes the sleeve via the connecting rod to cover the sharp blade.

44. The system of claim 41, wherein the sleeve comprises a conical tip and a slit into the conical tip, dividing the conical tip into two half covers that can bend away from each other, wherein translating the sleeve to a proximal position on the delivery tool causes the conical tip to open as the conical tip passes over the base of the attachment device and to close behind the base, wherein after the shaft has been used to drive the attachment device through a graft and into soft tissue, the closed conical tip sitting behind the base can be used to apply counterpressure to the base while the blade withdrawn from the attachment device.

45. The system of claim 41, wherein the attachment device includes at least two laterally opposed barbs pointing outward, and further wherein the attachment device has only the one leg.

46. The system of claim 41, wherein the sharp blade comprises a substantially flat piece with opposed first and second surfaces extending distally to a sharpened piercing tip, wherein the sharpened piercing tip of the sharp blade extends distally beyond the distal tip of first leg of the attachment device.

47. The system of claim 41, wherein the sleeve includes a static comb with one or more steps that can push the attachment device in distal direction, wherein the static comb pushes the attachment device into tissue during surgery.

48. (canceled)

49. The system of claim 47, wherein the static comb is in a fixed position with respect to the elongated shaft, and wherein the sleeve translates with respect to the static comb.

50. The system of claim 49, further comprising a dynamic comb fixed to the sleeve, wherein the dynamic comb pushes pre-loaded attachment devices in a distal direction down the sharp blade to position a subsequent device in position for delivery.

51-55. (canceled)
